(19) United States
(12) Patent Application Publication
(10) Pub. No.: US 2024/0100224 A1
(43) Pub. Date: Mar. 28, 2024

(54) TWO-FIELD COUPLING CROSSLINKED, INJECTABLE, MOLDABLE AND PRINTABLE GRANULAR HYDROGEL MATERIAL, PREPARATION METHOD THEREOF AND APPLICATION THEREOF

(71) Applicant: DALIAN UNIVERSITY OF TECHNOLOGY, Dalian, Liaoning (CN)

(72) Inventors: Huanan WANG, Dalian, Liaoning (CN); Kaiwen CHEN, Dalian, Liaoning (CN)

(21) Appl. No.: 18/263,046

(22) PCT Filed: May 19, 2021
(86) PCT No.: PCT/CN2021/094525

§ 371 Date: Jul. 26, 2023

(30) Foreign Application Priority Data
Jan. 29, 2021 (CN) 202110125753.5

(51) Int. Cl. A61L 27/52; A61L 24/00; A61L 24/10; A61L 27/22; A61L 27/56; A61L 31/04; A61L 31/14

(52) U.S. Cl. CPC A61L 27/52; A61L 24/0031; A61L 24/0036; A61L 24/104; A61L 27/222; A61L 27/56; A61L 31/045; A61L 31/145; A61L 31/146; A61L 2300/424; A61L 2400/04; A61L 2400/06; A61L 2430/02

(57) ABSTRACT

An injectable, moldable and printable gramular hydrogel material crosslinked by non-covalent and covalent bonds, a preparation method therefor, and applications thereof are provided. The material uses gelatin particles or particles that have core-shell structures as basic structural units, and forms a continuous and porous particle network by means of the reversible non-covalent crosslinking and covalent bond crosslinking of the particles. The gelatin particles or the particles that have core-shell structures form a continuous porous particle network by means of reversible self-assembly under the effect of the non-covalent bonds, thus achieving injectable, printable, moldable and self-healing properties. Furthermore, high strength granular hydrogels are formed by means of initiating covalent crosslinking. The material can be used as a drug-sustained release carrier, a tissue engineering scaffold and a tissue adhesive hemostatic material in the field of biomedicine.

### TECHNICAL FIELD

The present invention belongs to the field of material science and the field of biomedical materials, in particular to a non-covalent bond and covalent bond two-field coupling crosslinked, injectable, moldable and printable granular hydrogel material, and preparation method and application thereof.

### BACKGROUND

The rapid development of the economy in China stimulates continuous increasing of public demands for a high level of health care, and in addition, the increasing severe aging population problem promotes continuous growth of demand for human tissue and organ repair and reconstruction in clinical medicine. Therefore, in recent years, regenerative medicine, which uses tissue engineering as the main means to heal and reconstruct human tissue, has been developed rapidly, bringing hope for organ healing and regeneration clinically. A tissue engineering technique rebuilds tissue, in a manner of direct in vivo implantation or in vitro engineered culture, by using biomaterials as a scaffold, combining cells from an organ to-be-repaired or mesenchymal stem cells with multiple differentiation functions, and loading biological signaling factors (such as growth factor) that can regulate cell behavior. As a basis for tissue repairing and construction, biomaterial scaffolds provide spatial and mechanical support for cell reproduction and differentiation, and tissue reconstruction. An ideal scaffold material needs to have physical properties and chemical composition similar to Extracellular Matrix (ECM), providing cells with a microenvironment suitable for their growth and function, providing cells with physical properties including mechanical strength, microtopography, and adhesion to surrounding tissues, and further providing chemical signals such as cell adhesion sites, chemical composition, growth factor expression, and biodegradability, so as to accurately manipulate cell aggregation, reproduction, renewal and differentiation of cells and ultimately induce tissue regeneration.

Hydrogels are the most widely used matrix biomaterials for a bionic cell microenvironment. A typical hydrogel consists of hydrophilic polymers, which is water-rich similar to natural ECM, allowing diffusion of biomacromolecules through a porous gel network. Hydrogels can be regulated in stiffness by crosslinking degree and are easy to be chemically modified. Therefore, hydrogels are also one of the most important biomaterials in the field of tissue engineering and drug controlled release. However, traditional hydrogels are mostly composed of permanent covalent bonds or long-term stable strong physical bonds, the mechanical characteristics thereof are mainly elasticity, and in addition, the gel network with nanopores will inhibit spreading, migration and proliferation of cells, and is difficult to be reconstructed by cells, which is not an ideal material for bionic cell microenvironment. Traditional hydrogel materials are designed in a “top-down” means, that is, a scaffold material is constructed through mold forming and subtractive manufacturing by further triggering covalent crosslinking after forming of molding and subtractive manufacturing, so that the resulting material has simple structure, single composition and limited functions, and cannot be used for repairing human organs with complex structure, multi-component and multi-function. Moreover, traditional material systems using hydrogels as a tissue engineering scaffold do not have injectable and printable properties, and do not have good adhesion or integration with surrounding tissues.

More and more research has recently focused on designing hydrogel materials with shear-thinning and self-healing properties compared with hydrogels designed and prepared by permanent covalent bonding crosslinking mechanisms. The hydrogel network constructed by physical crosslinking of the hydrogel materials with shear-thinning and self-healing properties is reversible and exhibits shear-thinning characteristics when subjected to destructive shear, that is, the viscosity of the hydrogel materials decreases as the shear force increases, and when the external force disappears, physical crosslinking can occur immediately due to the reversibility between the molecular chains, so as to restore the structure and strength thereof (i.e., self-healing behavior). So the viscosity of the hydrogel materials is reduced under a high shear force during injection, and thus can be extruded, and the mechanical properties thereof is recovered by means of the self-healing properties, allowing the material to maintain its mechanical properties after being sheared and injected. Shear-thinning and self-healing hydrogel materials are currently being investigated in a variety of biomedical applications, including drug delivery, tissue regeneration and bio-three dimensional (3D) printing. In order to achieve shear-thinning and self-healing characteristics of hydrogel materials, a reversible interaction group is usually introduced on the polymer chains, so that the hydrogel materials can quickly recover their initial mechanical strength after experiencing damage at a high shear rate. However, introduction of reversible groups on polymer chains usually requires complex chemical modifications, which usually leads to the reduction of biocompatibility of hydrogel materials and makes it difficult to achieve further conversion applications in biomedical fields.

In addition, the gels are prone to phase separation since the interaction between the polymer chains is difficult to control when preparing gels with reversible interaction. Moreover, most reversible interactions, such as hydrogen bonds and ionic bonds, are often fragile in a physiological environment, resulting in the failure of gel formation. Therefore, shear-thinning and self-healing hydrogel materials that have excellent biocompatibility, simple synthesis and stable structure in physiological environments have a promising future in biomedical applications. Some colloidal gels exhibit shear-thinning and self-healing behaviors due to the reversibility of the interaction between physically “crosslinked” colloidal granules, and can be used as injectable materials for filling and repairing tissue defects or for 3D printing to build engineered scaffolds with complex structures. Self-healing ability allows the gel to quickly recover its strength and plasticize after injection, and ensures that the material maintains its structural and strength stability after implantation into a human body, facilitating the smooth progress of tissue repair.

On the other hand, a “bottom-up” material design concept has been increasingly valued in recent years compared to the traditional “top-down” design concept. The “bottom-up” concept is to construct scaffold or engineered tissue with an integral structure by chemical or physical interaction by using micro/nano sized granules, material modules or cellularized tissue blocks as basic units. Compared with traditional methods, the “bottom-up” method can construct biomaterials with controlled structure and components and more complex functions, and can realize biomaterials for mimicking complex structures and functions of human organs by means of technologies such as 3D printing and controlled self-assembly. Granular hydrogel is a new hydrogel material with a fine microstructure and a stable macrostructure based on the “bottom-up” design concept and using micro/nano sized granules as basic units. Unlike traditional hydrogels which consist of continuous polymer networks, colloidal gels are composed of discontinuous granules. Such colloidal gels use micro/nano granules as basic structural units and control interactions between the basic structural units, such as magnetic force, hydrophobic interaction, electrostatic force, steric resistance, etc., to induce their self-assembly to form scaffolds by means of bottom-up assembly strategy. The colloidal gels exhibits shear-thinning, self-healing and tissue surface self-adaptability due to the reversibility of the interaction between physically “crosslinked” colloidal granules. Moreover, the granular hydrogel is simple in synthesis and less prone to phase separation during mixing, and exhibits stable mechanical properties in a physiological environment. Therefore, granular hydrogel is ideal for shear-thinning, self-healing hydrogel in biomedical applications. However, the granular hydrogel has poor mechanical strength and is difficult to combine with tissue in practical applications, which makes it difficult to satisfy the basic requirements of human weight-bearing tissue/organ repair, further limiting its application prospects in medical fields such as tissue engineering. Therefore, it is a technical challenge in this field to improve the mechanical strength of colloidal gels while taking into account the advantages of shear-thinning, self-healing and tissue adaptability.

Currently, researchers mainly increase the mechanical strength of colloidal gels by adding hard nanoparticles or nanofibers to compound with granular gels. The addition of hard materials has been proved to increase the mechanical strength of granular gels. However, colloidal granules are still assembled by physical interaction and its disadvantage of poor integrity has not been alleviated. This disadvantage of poor integrity makes long-term structural stability of a colloidal gel system affected by factors such as pH, ion concentration, and fluid impact in a complex physiological environment, causing the colloidal granules that make up the hydrogel network to disperse or wander to other sites, which not only affects the property requirements of the granular gels as a tissue engineering scaffold, but also creates the possibility of other side effects caused by free granules. Therefore, how to maintain the structural integrity of granular gel materials after injection and molding is a challenge that must be addressed for medical applications of injectable granular gel materials. In summary, enhancing the mechanical properties of the granular gels and maintaining the structural stability of the granular gel network are the keys to achieve the application of granular hydrogel system in the field of regenerative medicine.

The present invention innovatively develops gelatin granules or gelatin core-shell granules as basic units. In the core-shell granules, a rigid core increases the mechanical strength of the colloidal granules and a flexible shell allows the granules to retain the high deformability and surface charge of a gelatin polymer phase, so that the reversible interaction (including electrostatic, hydrophobic and hydrogen bonding forces) can be established between the composite material granules through the gelatin polymer phase, so as to maintain the self-healing ability of the colloidal gel network, and further increase dense stacking degree and volume fraction of the colloid, realizing enhancement of the colloidal gel. Further, the composite of rigid core granules also endows the colloidal gel with additional functionalities of osteogenesis, photothermal property, magnetic responsiveness, etc. A new material design concept of further introducing covalent bond crosslinked groups in a gelatin phase to construct a colloidal gel with high strength and self-healing ability lays a theoretical foundation for the wide application of colloidal gel-like materials in the biomedical field.

### SUMMARY

In order to give consideration to shear-thinning and self-healing properties and mechanical properties of macroscopic stability and high strength of granular gels, the present invention provides an injectable high-strength granular hydrogel material based on a covalently crosslinkable gelatin granular gel, and a preparation method and application thereof. In the present invention, chemical crosslinking between gelatin granules is achieved by grafting covalently crosslinkable groups on surfaces of the granules, so as to form a granular hydrogel with high stability and high strength.

For solving the above technical problem, the present invention uses the following technical solutions.

A first aspect of the present invention provides a non-covalent bond and covalent bond two-field coupling crosslinked, injectable, moldable and printable granular hydrogel material. The granular hydrogel material uses a gelatin granule as a basic structural unit to form a continuous and porous granular network by means of reversible non-covalent crosslinking and covalent bond crosslinking between the gelatin granules, and the gelatin granules form the continuous and porous granular network by means of reversible self-assembly under the action of the non-covalent bonds so as to achieve injectable, printable, moldable and self-healing properties, and the high-strength granular hydrogel is further formed by initiating covalent bond crosslinking. The gelatin granules have a size ranging from 20 nm to 50 μm, a volume fraction of the gelatin granules in a total volume of the granular hydrogel material is 2 v/v % to 100 v/v %, a degree of substitution of covalent crosslinking groups on gelatin macromolecular chains in the gelatin granules is 5% to 80%, the continuous and porous granular network has a pore size of 0.1 μm to 100 μm and is formed by connecting granules through polymer chains, and the obtained granular hydrogel material has a compressive elastic modulus of 0.5 kPa to 500 kPa.

A second aspect of the present invention provides a non-covalent bond and covalent bond two-field coupling crosslinked, injectable, moldable and printable granular hydrogel material of a core-shell structure. The granular hydrogel material of a core-shell structure uses a granule of a core-shell structure as a basic structural units to form a continuous and porous granular network by means of reversible non-covalent bonds and covalent bonds between the granules, the gelatin granules of a core-shell structure form the continuous and porous granular network by means of reversible self-assembly under the action of the non-covalent bonds so as to achieve injectable, printable and moldable properties, and the high-strength granular hydrogel is further formed by initiating covalent bond crosslinking on surfaces of the granules to enhance curing. Shell layer granules of the granules of a core-shell structure have a size ranging from 50 nm to 50 μm and core layer granules of that have a size ranging from 10 nm to 1 μm, a volume fraction of the granules of a core-shell structure in a total volume of the granular hydrogel material is 2 v/v % to 100 v/v %, the obtained continuous and porous granular network has a pore size of 0.1 μm to 100 μm, and the obtained granular hydrogel material has an elastic modulus of 10 kPa to 1000 kPa.

A third aspect of the present invention provides a preparation method of the foregoing non-covalent bond and covalent bond two-field coupling crosslinked, injectable, moldable and printable granular hydrogel material. When covalent bond is crosslinked by free radical polymerization of granule surface groups, the preparation method includes the following steps of:
(1) Dissolving gelatin in an aqueous solution at 30° C. to 60° C. to obtain a gelatin aqueous solution with a concentration of 0.1 w/v % to 10 w/v %.
(2) Adding a compound reacting with the hydroxyl and amino groups of on a gelatin polymer chain into the gelatin aqueous solution to obtain a modified gelatin polymer solution containing a modified gelatin polymer compound of formula III, where the compound reacting with the a hydroxyl group and an amino groups of a gelatin polymer chain is a compound shown as formula I or formula II, preferably acrylic anhydride, acryloyl chloride, methacrylic anhydride, methacryloyl chloride, ethyl acrylic anhydride, ethyl acryloyl chloride, hydroxy acrylic anhydride, hydroxy acryloyl chloride, isobornyl acrylic anhydride, isobornyl acryloyl chloride, allyl isocyanate anhydride and allyl isocyanate chloride.

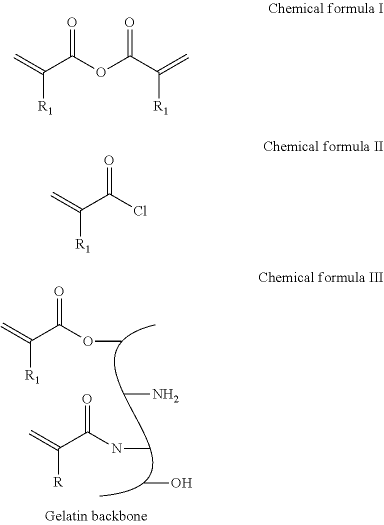

In formula I, formula II and formula III, R and R₁ are selected from the group consisting of hydrogen, halogen atom, hydroxyl, sulfhydryl, amine group, nitro group, cyano group, aldehyde group, keto group, ester group, amide group, phosphonic acid group, phosphonate group, sulfonic acid group, sulfonate group, sulfone group, sulfoxide group, aryl group and alkyl group. A substituent of the aryl group is hydrogen, hydroxyl, amino or methyl, and the number of the substituent is 1-5. The alkyl group is preferably C₁₋₁₀ alkyl group, more preferably C₁₋₄ alkyl group. The aryl group is preferably phenol. The halogen atom is fluorine atom, chlorine atom, bromine atom, or iodine atom.
(3) Adding a polar organic solvent into the modified gelatin polymer solution until the modified gelatin polymer compound is precipitated out, followed by washing the modified gelatin polymer compound and re-dissolving with an aqueous solution at 30° C. to 60° C. to obtain a modified gelatin aqueous solution with a concentration of 0.1 w/v % to 20 w/v %.
(4) Adjusting a pH value of the modified gelatin aqueous solution to 1-5 or 9-14, and dripping the polar organic solvent into the modified gelatin aqueous solution to obtain a covalently crosslinkable gelatin granular suspension, where a volume of the added polar organic solvent is 1 time to 10 times of a volume of the modified gelatin aqueous solution; carrying out crosslinking reaction for 1 hour to 12 hours at a normal temperature to obtain a covalently crosslinkable gelatin granular dispersion liquid after washing, and freeze-drying the granular dispersion liquid to obtain a modified gelatin granular powder.
(5) Blending the modified gelatin granular powder with the aqueous solution to obtain a colloidal gel, and adding a chemical initiator or a photo-crosslinking agent to initiate free radical polymerization, so that the modified gelatin granules are covalently crosslinked to further obtain mechanically-enhanced non-covalent bond and covalent bond composite crosslinked gelatin granules assembled to form the granular hydrogel material.

A fourth aspect of the present invention provides another preparation method of the foregoing non-covalent bond and covalent bond two-field coupling crosslinked, injectable, moldable and printable granular hydrogel material. When crosslinking is achieved by click chemistry of granule surface groups, the preparation method includes the following steps of:
(1) Dissolving gelatin in an aqueous solution at 30° C. to 60° C. to obtain a gelatin aqueous solution with a concentration of 0.1 w/v % to 10 w/v %.
(2) Adding a compound capable of carrying out amidation reaction with carboxyl or amino on a surface of gelatin into the gelatin solution to respectively obtain a solution containing modified gelatin polymer compound A or a solution containing modified gelatin polymer compound B with a structural formula according with any one of formulas VI or VII. The compound capable of carrying out amidation reaction with carboxyl or amino on the surface of the gelatin is preferably a compound shown as chemical formula IV or V, and is preferably azide succinimide/alkyne ethylamine, azide imine/propargylamine, mercaptoethylamine/ethyleneimine, and 2-amino ethanethiol/ethyleneimine.

In formula IV, formula V and formula VI, R₂ is a combination of azide/alkyne, sulfhydryl/double bond, thiol/alkene or diene/mono olefinic bond, and R₃ is selected from the group consisting of hydrogen, halogen atom, hydroxyl, sulfhydryl, amine, nitro group, cyano group, aldehyde group, keto group, ester group, amide group, phosphonic acid group, phosphonate group, sulfonic acid group, sulfonate group, sulfone group, sulfoxide group, aryl group and alkyl group. A substituent of the aryl group is hydrogen, hydroxyl, amino or methyl, and the number of the substituent is 1-5. The alkyl group is preferably C₁₋₁₀ alkyl group, more preferably C₁₋₄ alkyl group. The aryl group is preferably phenol. The halogen atom is fluorine atom, chlorine atom, bromine atom or iodine atom.

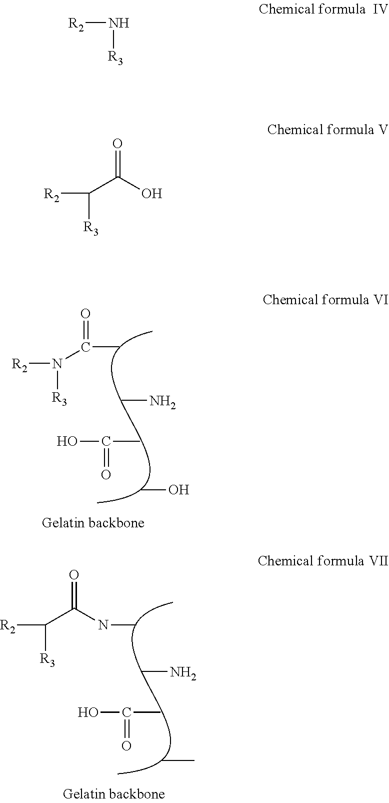

(3) Adding a polar organic solvent into each of the solution containing modified gelatin polymer compound A and the solution containing modified gelatin polymer compound B until a modified gelatin polymer compound is precipitated out, followed by washing the modified gelatin polymer compound and re-dissolving with an aqueous solution at a temperature of 30° C. to 60° C. to obtaining two modified gelatin aqueous solutions with a concentrations of 0.1 w/v %-20 w/v %, respectively.
(4) Adjusting a pH value of each of the two modified gelatin aqueous solutions to 1-5 or 9-14, and dripping the polar organic solvent into each of the two modified gelatin aqueous solutions to obtain two covalently crosslinkable gelatin granular suspensions, where a volume of the added polar organic solvent is 1 time to 10 times of a volume of the modified gelatin aqueous solution, and carrying out crosslinking reaction for 1 hour to 12 hours at a normal temperature to obtain two click chemical crosslinkable gelatin granular dispersion liquids after washing, and separately freeze-drying the two granular dispersion liquids to obtain a modified gelatin granular powder A and a modified gelatin granular powder B.
(5) Uniformly blending the two gelatin granular powders A and B having click chemical group combinations at a ratio of 0.1-10, followed by rapidly blending with an aqueous solution uniformly to obtain a colloidal gel and stand for 2 min to 60 min to obtain the covalent crosslinked granular hydrogel material by means of covalent crosslinking between the gelatin granules in a click chemistry reaction.

A fifth aspect of the present invention provides a preparation method of the foregoing non-covalent bond and covalent bond two-field coupling crosslinked, injectable, moldable and printable granular hydrogel material of a core-shell structure. When covalent bond is crosslinked by free radical polymerization of granule surface groups, the preparation method includes the following steps of:
(1) Dissolving gelatin in an aqueous solution at 30° C. to 60° C. to obtain a gelatin aqueous solution with a concentration of 0.1 w/v % to 10 w/v %.
(2) Adding a compound reacting with the hydroxyl and amino groups on a gelatin polymer chain into the gelatin aqueous solution to obtain a modified gelatin polymer solution containing a modified gelatin polymer compound of formula III, where the compound reacting with the hydroxyl and amino groups on a gelatin polymer chain is preferably a compound shown as formula I or formula II, more preferably acrylic anhydride, acryloyl chloride, methacrylic anhydride, methacryloyl chloride, ethyl acrylic anhydride, ethyl acryloyl chloride, hydroxy acrylic anhydride, hydroxy acryloyl chloride, isobornyl acrylic anhydride, isobornyl acryloyl chloride, allyl isocyanate anhydride and allyl isocyanate chloride.

In formula I, formula II and formula III, R and R₁ are selected from the group consisting of hydrogen, halogen atom, hydroxyl, sulfhydryl, amine group, nitro group, cyano group, aldehyde group, keto group, ester group, amide group, phosphonic acid group, phosphonate group, sulfonic acid group, sulfonate group, sulfone group, sulfoxide group, aryl group and alkyl group. A substituent of the aryl group is hydrogen, hydroxyl, amino or methyl, and the number of the substituent is 1-5. The alkyl group is preferably C₁₋₁₀ alkyl group, more preferably C₁₋₄ alkyl group. The aryl group is preferably phenol. The halogen atom is fluorine atom, chlorine atom, bromine atom or iodine atom.

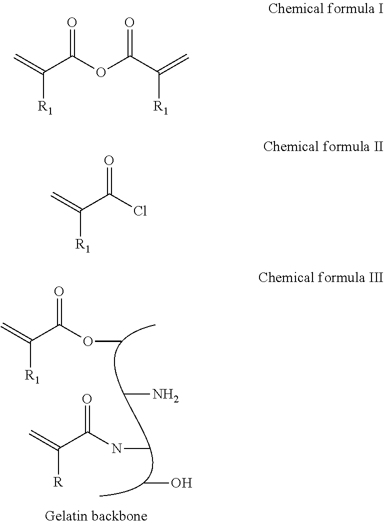

(3) Adding a polar organic solvent into the modified gelatin polymer solution until a modified gelatin polymer compound is precipitated out, followed by washing the modified gelatin polymer compound and re-dissolving with a suspension containing 0.1 w/v % to 50 w/v % of rigid nanoparticles at 30° C. to 60° C. to obtain a modified gelatin/rigid nanoparticle suspension with a concentration of 0.1 w/v % to 10 w/v % of gelatin capable of free radical polymerization crosslinking.
(4) Adjusting a pH value of the modified gelatin/rigid nanoparticle suspension to 1-5 or 9-14, and dripping the polar organic solvent into the modified gelatin/rigid nanoparticle suspension to obtain a modified core-shell structural gelatin granular suspension, where a volume of the added polar organic solvent is 1 time to 10 times of a volume of the modified gelatin/rigid nanoparticle suspension, and carrying out crosslinking reaction for 1 hour to 12 hours at a normal temperature to obtain a modified gelatin core-shell granular dispersion liquid after washing, and freeze-drying the granular dispersion liquid to obtain a modified gelatin core-shell granular powder.

(5) Blending the modified gelatin core-shell granular powder with the aqueous solution to obtain a colloidal gel, and adding a chemical initiator or a photo-crosslinking agent to initiate free radical polymerization, so that the modified gelatin granules are covalently crosslinked to further obtain mechanically-enhanced non-covalent bond and covalent bond composite crosslinked gelatin granules of a core-shell structure assembled to form the granular hydrogel material.

A sixth aspect of the present invention provides another preparation method of the foregoing non-covalent bond and covalent bond two-field coupling crosslinked, injectable, moldable and printable granular hydrogel material of a core-shell structure. When crosslinking is achieved by click chemistry of granule surface groups, the preparation method includes the following steps of:
(1) Dissolving gelatin in an aqueous solution at 30° C. to 60° C. to obtain a gelatin aqueous solution with a concentration of 0.1 w/v % to 10 w/v %.
(2) Adding a compound capable of carrying out amidation reaction with the carboxyl group or amino group on a surface of gelatin into the gelatin solution to respectively obtain a solution containing modified gelatin polymer compound C or a solution containing modified gelatin polymer compound D with a structural formula according with any one of formulas VI or VII, where the compound capable of carrying out amidation reaction with the carboxyl group or the amino group on the surface of the gelatin is preferably a compound shown as formula IV or V, and is preferably azide succinimide/alkyne ethylamine, azide imine/propargylamine, mercaptoethylamine/ethyleneimine, and 2-amino ethanethiol/ethyleneimine.

In formula IV, formula V, formula VI, and formula VII, R₂ is a combination of azide/alkyne, sulfhydryl/double bond, thiol/alkene or diene/mono olefinic bond, and R₃ is selected from the group consisting of hydrogen, halogen atom, hydroxyl, sulfhydryl, amine group, nitro group, cyano group, aldehyde group, keto group, ester group, amide group, phosphonic acid group, phosphonate group, sulfonic acid group, sulfonate group, sulfone group, sulfoxide group, aryl group and alkyl group. A substituent of the aryl group is selected from hydrogen, hydroxyl, amino or methyl, and the number of the substituent is 1-5. The alkyl group is preferably C₁₋₁₀ alkyl group, and more preferably C₁₋₄ alkyl group. The aryl group is preferably phenol. The halogen atom is fluorine atom, chlorine atom, bromine atom or iodine atom.

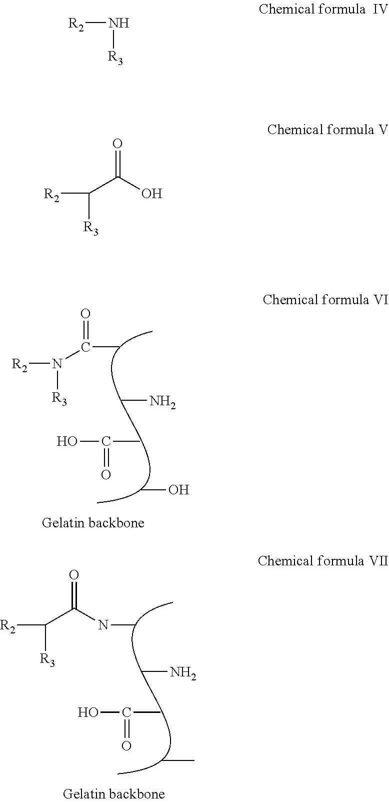

(3) Adding a polar organic solvent into each of the solution containing modified gelatin polymer compound C and the solution containing modified gelatin polymer compound D until a modified gelatin polymer compound is precipitated out, followed by washing the modified gelatin polymer compound and re-dissolving with a suspension containing 0.1 w/v % to 50 w/v % of rigid nanoparticles at 30° C. to 60° C. to obtain two modified gelatin/rigid nanoparticle suspensions with a click chemical crosslinkable gelatin concentration of 0.1 w/v % to 10 w/v %, respectively.
(4) Adjusting a pH value of each of the two modified gelatin/rigid nanoparticle suspensions to 1-5 or 9-14, and dripping the polar organic solvent into each of the two modified gelatin/rigid nanoparticle suspensions to obtain two modified core-shell structural gelatin granular suspensions, where a volume of the added polar organic solvent is 1 time to 10 times of a volume of the modified gelatin/rigid nanoparticle suspension, and carrying out crosslinking reaction for 1 hour to 12 hours at a normal temperature to obtain two modified gelatin core-shell granular dispersion liquids after washing, and separately freeze-drying the granular dispersion liquids to obtain two modified gelatin core-shell granular powders C and D.
(5) Uniformly blending the two gelatin core-shell granular powders C and D having click chemical group combinations at a ratio of 0.1-10, followed by rapidly blending with an aqueous solution uniformly to obtain a colloidal gel and stand for 2 min to 60 min to obtain the covalent crosslinked granular hydrogel material by means of covalent crosslinking between the gelatin granules in a click chemistry reaction.

In the above technical solutions, further, the polar organic solvent is methanol, ethanol, isopropanol, butanol, acetone, acetonitrile or tetrahydrofuran. The photo-crosslinking agent is at least one of glutaraldehyde, glyceraldehyde, genipin, tyrosinase and 1-(3-dimethylaminopropyl)-3-ethyl carbodiimide hydrochloride/N-hydroxysuccinimide. The chemical initiator for inducing polymerization may be benzoyl peroxide, tert-butyl hydroperoxide, ammonium persulfate/tetramethylimide (a mass ratio of ammonium persulfate to tetramethylimide is 0.5-100:1, more preferably 1:1), and a concentration of the chemical initiator has is 0.0001 g/mL(w/v) to 0.02 g/mL(w/v).

In the above technical solutions, further, the rigid nanoparticles are selected from at least one of silicon dioxide nanoparticles, lithium magnesium silicate nanoparticles, nano-clay particles, hydroxyapatite nanoparticles, iron oxide magnetic nanoparticles, barium titanate nanoparticles, graphene nanosheets, carbon nanotubes, bioglass nanoparticles, black phosphorus nanosheets, silk fibroin nanoparticles, polylactic acid nanoparticles, polyethylene nanoparticles and polystyrene nanoparticles, and the rigid nanoparticles have a size ranging from 10 nm to 50 μm.

In the above technical solutions, further, the aqueous solution is a solution containing a bioactive substance, and the bioactive substance is vitamins, amino acids, mineral elements, microecological regulators, growth factors or blood. In step (5), the aqueous solution is directly blended with at least one of rigid granules of hydroxyapatite, silicon dioxide, bioglass, manganese dioxide, carbon quantum dots, graphene, montmorillonite, black phosphorus, silk fibroin, polylactic acid, etc., and the rigid granules have a size ranging from 10 nm to 1 μm.

A seventh aspect of the present invention provides the foregoing granular hydrogel material serving as a carrier or scaffold of a medicine component, which is used for repairing and filling wounds or defects of bone tissue, cartilage tissue, muscle and blood vessels. The medicine component is at least one of vitamins, amino acids, mineral elements, microecological regulators, growth factors, protein macromolecular medicines, protein micromolecular medicines and living cells.

An eighth aspect of the present invention provides an application of the foregoing granular hydrogel material as a bone repair filler material. During application, covalently crosslinkable gelatin colloidal granules formed under the action of non-covalent bonds are blended with an aqueous solution to obtain a granular gel having a mass fraction of 5% to 50% and a volume fraction of 10% to 120%, the granular gel is directly injected into a bone defect area, and then a high-strength bone filling material is obtained by initiating covalent crosslinking between the gelatin colloidal granules.

A ninth aspect of the present invention provides an application of the foregoing granular hydrogel material as a bioprinting ink for living cell-laden printing, where during application, covalently crosslinkable gelatin colloidal granules formed under the action of non-covalent bonds are blended with an aqueous solution to obtain a granular gel having a mass fraction of 5% to 50% and a volume fraction of 10% to 120%, then the granular gel is mixed with a cell suspension to obtain cell-laden granular gel having a volume fraction of 10% to 100% as a bioprinting ink, the bioprinting ink is extruded or subject to three dimensional (3D) ink-jet printing to obtain a scaffold of a 3D structure, then a high-strength cell-laden printed scaffold is obtained by initiating covalent crosslinking between the gelatin colloidal granules after printing.

A tenth aspect of the present invention provides an application of the foregoing granular hydrogel material as a tissue adhesive gel material. The gelatin granules have a size less than 10 μm and an adhesive strength between the granular hydrogel and tissue is 5 kPa to 100 kPa.

Covalently crosslinkable gelatin granules or granules of a core-shell structure prepared according to the foregoing third aspect or fifth aspect are blended with a photo-crosslinking agent and then injected into a tissue injury site in vivo, and covalent crosslinking between the gelatin granules is achieved by light irradiating a gel surface to generate covalent bond polymerization. Alternatively, a chemical initiator is blended with a gelatin composite gel and then injected into a tissue injury site in vivo, then covalent crosslinking is achieved after 1 min-30 min, and a stable adhesion is formed due to a mechanical interlocking effect between the gel material and tissue.

Alternatively, click chemical crosslinkable gelatin granules or granules of a core-shell structure prepared according to the foregoing fourth aspect or sixth aspect are blended with an aqueous solution and then directly injected into a tissue injury site in vivo, then click chemical covalent crosslinking is achieved after 1 min to 30 min, and a stable adhesion is formed due to a mechanical interlocking effect with tissues.

An eleventh aspect of the present invention provides an application of the granular hydrogel material as a post-operative anti-adhesion gel. An injectable tissue adhesive gel is injected into a post-operative anti-adhesion site, and the injectable tissue adhesive gel stably covers the injury site after covalent crosslinking. The granular hydrogel acts as a barrier to effectively prevent adhesion between tissues after surgery.

A twelfth aspect of the present invention provides a rapid hemostatic sealant, obtained by freeze-drying the gelatin granular suspension prepared through the foregoing preparation method. The gelatin granular powder prepared according to the third aspect or the fifth aspect is uniformly blended with a chemical crosslinking agent powder or a photo-crosslinking agent powder, and then is directly sprayed on a bloody wound surface, and covalent crosslinking between the gelatin granules is achieved by standing directly or photo-induced polymerization after the powder fully absorbs oozing blood. Alternatively, the powder containing click chemical crosslinkable gelatin granules prepared according to the fourth aspect or the sixth aspect is blended and then directly sprayed on a bloody wound surface, and covalent crosslinking between the gelatin granules is achieved by standing directly after the powder fully absorbs oozing blood. The granules form stable adhesion with tissue after crosslinking.

A thirteenth aspect of the present invention provides applications of the granular hydrogel material in preparation of a skin repairing material or medicine for post-operative wound surface sealing, in preparation of an oral ulcer material or medicine for post-operative wound surface sealing, in preparation of a intestinal leakage occlusion material or medicine for tissue fluid leakage occlusion, in preparation of a surgical suture material or medicine for tissue fluid leakage occlusion, in preparation of a liver hemostatic material or medicine, in preparation of bone section hemostatic material or medicine, in preparation of an arterial hemostatic material or medicine, in preparation of a heart hemostatic material or medicine, in preparation of a cartilage repair material or medicine as tissue engineering scaffold material, in preparation of a bone repair material or medicine as tissue engineering scaffold material, and in preparation of a bone/cartilage composite defect repair material or medicine as tissue engineering scaffold material.

The present invention has the following beneficial effects.

1. The present invention provides a gel composed of covalently crosslinkable gelatin granules. The gel features shear-thinning and self-healing due to reversible interaction between the granules when the gel is not covalently crosslinked. After further achieving injection or printing properties, the mechanical strength of the gel is enhanced through covalent bonds crosslining on the surfaces of the granules. This property makes the gel have broad application prospects in the fields of minimally invasive implant materials, artificial extracellular matrices, 3D bioprinting ink, etc.

2. The covalently crosslinkable gelatin granular gel prepared by the present invention has excellent properties of injectability, self-healing and plasticity, and granular hydrogel with high mechanical strength and structural stability can also be obtained by means of covalent crosslinking between the granules. By regulating a mass fraction of the gel, a storage modulus of the covalent crosslinking group grafted degree gel can be regulated between 10 kPa and 500 kPa, which is much higher than that of the traditional granular gel formed by physical interaction and ensures the structural integrity and high mechanical strength of the gel after implantation.

3. The gelatin granular gel provided in the present invention can be used as a platform for compounding with other rigid nanoparticles, so as to prepare a gel with complex functionality and high mechanical strength. A core-shell structural colloidal gel with gelatin as a shell is also provided in the present invention for the first time, which can prepare core-shell structure granules of different rigid nanoparticles while guaranteeing excellent mechanical properties of the gel, so as to provide important help for richer functionality of the covalent crosslinked colloidal hydrogel. Moreover, as for the covalently crosslinkable gelatin granular gel, a novel one-step method for preparing chemically modified gelatin granules is achieved by means of process optimization. The method is simple in process and high in yield, and is beneficial to scale-up process production of the covalently crosslinkable gelatin granular gel.

4. Compared to a traditional covalently crosslinked hydrogel (such as methacrylate gelatin (GelMA)) based on gelatin polymers, in the present invention, a gelatin polymer is pre-prepared into gelatin granules and then an injectable and self-healing granular gel is formed by means of granules assembly. In contrast, a chemically crosslinked gelatin polymer hydrogel only shows an injectable property in a low temperature environment, and the gel will be transformed into liquid again with the increase in temperature once entering a human body environment, making it difficult for an implant material to stay on tissue. 3D bioprinting used such gel must be achieved by apparatuses with fine temperature-controlled printing systems, which is inconvenient for extensive use of the material. In contrast, the covalently crosslinkable gelatin granular gel provided in the present invention is not influenced by an environment temperature during injection and printing. Meanwhile, the mechanical strength of the covalently crosslinked hydrogel formed by pre-preparing granules from gelatin followed by assembling is much higher than that of a gelatin polymer hydrogel with the same mass fraction. This makes the gelatin granular hydrogel have more extensive application advantages.

5. The chemically modified gelatin granular gel in the present invention further has excellent biocompatibility and biodegradability, which can be used as a controlled-release carrier of bioactive protein medicine (such as a growth factor for inducing tissue regeneration) for medicine sustained-release. Moreover, the chemically modified gelatin granular gel can be used as a cell three-dimensional matrix to achieve construction of a cell three-dimensional environment and support growth and reproduction of cells and other function, and can be used as a cell-laden three-dimensional injectable gel scaffold and a 3D printed scaffold for applications of tissue engineering and regenerative medicine. For bioink applications for cell printing: cells are usually blended directly with the granular gel, and a mixing process requires a high shear force, which easily destroys the cells and causes low cell viability. In order to ensure that the cells are subjected to a less shear force, it is necessary to use a granular gel with a lower volume fraction for cell-laden printing, which will lead to a significant decrease in printing formability, strength and structural integrity of the scaffold. In the present invention, by means of covalent crosslinking of gelatin granules, it is possible to use a granular gel with a lower concentration for printing. When the cells are mixed and printed, the shear force on the cells is reduced, a cell survival rate is increased, and a structural stability of the scaffold is guaranteed. Therefore, the printed scaffold based on the colloidal gel can be further implanted into a bearing portion in vivo, greatly expanding an application prospect of the printed scaffold.

6. The chemically modified gelatin granular gel in the present invention can be used as a wet tissue adhesive for wound hemostasis and tissue adhesion. At present, commercial available tissue adhesives mainly make contact with tissue by means of a crosslinkable hydrogel precursor solution and then interact with the tissue by initiating covalent crosslinking. However, such adhesives based on polymer covalent crosslinking mainly make contact with the tissue in a form of a solution, which is easy to flow on a dynamic tissue surface and difficult to stably stay on a specific area of the tissue surface. To address this issue, currently commercial available tissue adhesives such as Bioglue® and Preveleak® have addressed staying of the adhesive at the tissue site by increase the viscosity of pre-polymerized liquid in their latest generation of products to solve the problem of retention of the adhesive at the tissue site. However, such pre-polymerized liquid with increased viscosity is still easy to be dispersed in a large body fluid environment and a rapid bleeding environment, resulting in difficulty for the adhesive to stay at a specific tissue site. For the injectable and curable colloidal gel tissue adhesive prepared in the present invention, since a pre-polymerization precursor is not a solution but a shear-thinning and self-healing gel, the gel is stably combined by means of electrostatic interaction between granule units, making the gel stable in a liquid environment, and moreover, the reversible electrostatic action enables the gel to adapt to various kinds of irregular tissue. Most of the current tissue adhesives are composed of a single polymer network by means of covalent interactions, resulting in limited mechanical strength of the hydrogels. The granular gel-based tissue adhesives in the present invention are composed of a granular network and a covalent network, which have high mechanical strength.

7. The chemically modified gelatin granular gel in the present invention can be used as a post-operative anti-adhesion gel for preventing tissue adhesion at a post-operative wound. At present, post-operative anti-adhesion products used clinically are mainly anti-adhesion films, which can form a physical barrier between wound tissue and normal tissue. In practice, however, covering a film material on tissue is difficult, and the film material is difficult to use on tissue having an irregular surface or severely folded (for example, large blood vessels of a heart and small intestine), and moreover, interaction between the film material and the tissue is weak, making the film material easy to fall off from a surface of the tissue. Thus, despite these sheet film barriers are used clinically, there is still a risk that any uncovered tissue will form adhesions. The injectable and curable colloidal gel tissue anti-adhesion material in the present invention can be combined with target tissue in a minimally invasive manner, can adapt to tissue of various shapes, and is not prone to fall off due to stable combination between the cured gel and the tissue.

8. The chemically modified gelatin granular freeze-dried powder prepared by the present invention can quickly form granular gel having stable interaction with tissue after absorbing blood or tissue fluid, and the granular gel can further form covalent crosslinking and stable adhesion to the tissue, such that stable hemostasis and tissue adhesion are achieved. Compared with traditional hemostatic powder which stops bleeding by physical means, the covalently crosslinked granular gel based on hemostasis and tissue adhesion can implement rapid hemostasis and wound seal in a dynamic bleeding process, greatly expanding applications of the hemostatic powder material.

### BRIEF DESCRIPTION OF THE DRAWINGS

FIG. 1 shows surface charge of gelatin colloidal granules with different proportions of acrylic anhydride to gelatin in Embodiment 1 under different pH conditions.

FIG. 2 shows transmission electron microscopy images of gelatin granules with different proportions of acrylic anhydride to gelatin in Embodiment 1.

FIG. 3 shows scanning electron microscopy images of gelatin granules with different proportions of acrylic anhydride to gelatin in Embodiment 1.

FIG. 4 shows shear-thinning properties of a gelatin granular gel in Embodiment 1, where FIG. 4 panel A is change curves of apparent viscosity of gelatin granular gel as a shear rate, and FIG. 4 panel B is an injectable optical image of the gelatin granular gel.

FIG. 5 shows scanning electron microscopy image of a gelatin granular gel after covalent crosslinking in Embodiment 1.

FIG. 6 shows transmission electron microscopy images of silicon dioxide/methacrylate gelatin core-shell granules prepared by adding silicon dioxide granules of 1 mg/ml (a), 10 mg/ml (b), and 20 mg/ml (c) in Embodiment 5.

FIG. 7 shows transmission electron microscopy images of Embodiment 6, where FIG. 7 panel A is a transmission electron microscopy image of ferroferric oxide nanoparticles prepared in Embodiment 6, and FIG. 7 panel B and FIG. 7 panel C are transmission electron microscopy images of ferroferric oxide/methacrylate gelatin core-shell granules.

FIG. 8 shows transmission electron microscopy images of Embodiment 7, where FIG. 8 panel a is a transmission electron microscopy image of mesoporous bioglass granules prepared in Embodiment 7, and FIG. 8 panel b is a transmission electron microscopy image of mesoporous bioglass/methacrylate gelatin core-shell granules.

FIG. 9 shows transmission electron microscopy images of Embodiment 8, where FIG. 9 panel a is a transmission electron microscopy image of hydroxyapatite prepared in Embodiment 8, and FIG. 9 panel b and FIG. 9 panel c are transmission electron microscopy images of hydroxyapatite/methacrylate gelatin core-shell granules.

FIG. 10 shows compressive stress-strain curves of the gelatin granular gel before and after covalent crosslinking in Embodiment 9.

FIG. 11 shows mechanical properties of a covalently crosslinked gelatin granular gel in Embodiment 10 and a covalently crosslinked gelatin polymer hydrogel in Comparative example 1, where and FIG. 11 panel a is compressive test stress-strain curves, and FIG. 11 panel b is tensile test stress-strain curves.

FIG. 12 shows a factor release curve of a covalently modified gelatin granular hydrogel in Embodiment 11.

FIG. 13 is a phase diagram printed at different temperatures of a covalently crosslinked granular gel in Embodiment 1.

FIG. 14 shows printed images of gelatin granular gels with different covalent groups grafted in Embodiment 12.

FIG. 15 shows optical images of a printing process and a printed tissue structure of a covalently crosslinked granular gel in Embodiment 12.

FIG. 16 shows fluorescence images of cells on a surface of a printed scaffold of a gelatin granular hydrogel in Embodiment 13.

FIG. 17 shows fluorescence images of cells in a covalently modified gelatin granular hydrogel as three dimensional bioprinting ink in Embodiment 14.

FIG. 18 shows optical images of Embodiment 15, where FIG. 18 panel a is optical images of a covalently crosslinked gelatin granular gel tissue adhesive in Embodiment 15 injected and staying on a rat heart, and FIG. 18 panel b is optical images of a liquid adhesive in Comparative example 7 injected and staying on a rat heart.

FIG. 19 shows optical images of a bonding process of fractured ex vivo heart tissue by means of a covalently crosslinked gelatin granular gel tissue adhesive in Embodiment 16.

FIG. 20 shows optical images of stable adhesion of a covalently crosslinked gelatin granular gel tissue adhesive in Embodiment 16 to an isolated heart, liver and stomach.

FIG. 21 shows a comparison of adhesive strength of a granular gel adhesive in Embodiment 17 and a commercial available fibrin glue in Comparative example 7.

FIG. 22 shows an application of covalently modified gelatin granular powder as a hemostatic material in Embodiment 18.

FIG. 23 shows optical images of a granular gel used for post-operative anti-adhesion of a liver injury in Embodiment 19.

FIG. 24 shows hematoxylin-eosin (HE) stained sections of a granular gel used for a post-operative liver injury in Example 19.

### DETAILED DESCRIPTION OF THE EMBODIMENTS

The present invention is described in further detail below in conjunction with particular embodiments without limiting the present invention in any manner.

### Embodiment 1

1. 5 g of gelatin powder of type A was dissolved in 100 mL of deionized water at 50° C. to obtain a gelatin solution. 0.0625 g, 0.125 g, 0.25 g, 0.5 g and 2 g of methacrylic anhydride were separately added into the gelatin solution for reacting for two hours at a high temperature to obtain a reaction solution, and a nucleophilic substitution reaction occured between the methacrylic anhydride and a free amino group on a protein molecular chain and equimolar methacrylic acid was produced. A pH value of the reaction solution was adjusted to 7 with hydrochloric acid, followed by adding acetone twice a volume of the reaction solution to destroy a hydration layer on a surface of a protein molecule, and methacrylate gelatin (GelMA) was precipitated out. The obtained methacrylate gelatin was repeatedly washed with deionized water followed by freeze-drying to obtain a freeze-dried methacrylate gelatin sample. A grafting degree of an amino group on a surface of gelatin was measured by means of H nuclear magnetic resonance. The grafting degree computed according to a change of a corresponding group spectrum is shown in Table 1.

TABLE 1

| Mass of methacrylic anhydride/g | 0.125 g | 0.25 g | 0.5 g | 2 g |
|---|---|---|---|---|
| Grafting degree of amino group | 15% | 32% | 59% | 87% |

2. Preparation of GelMA Nanoparticles

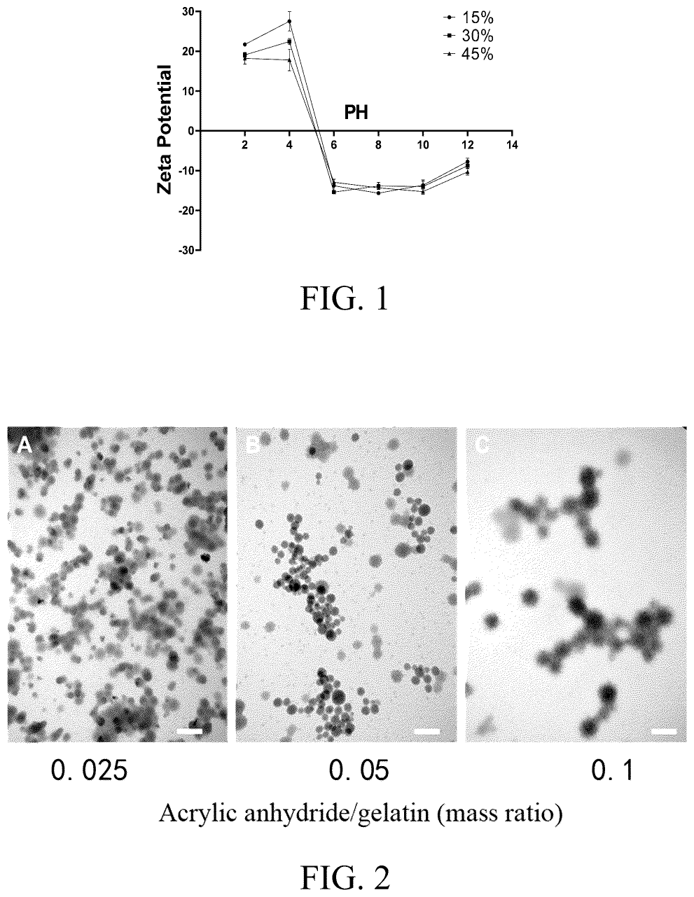

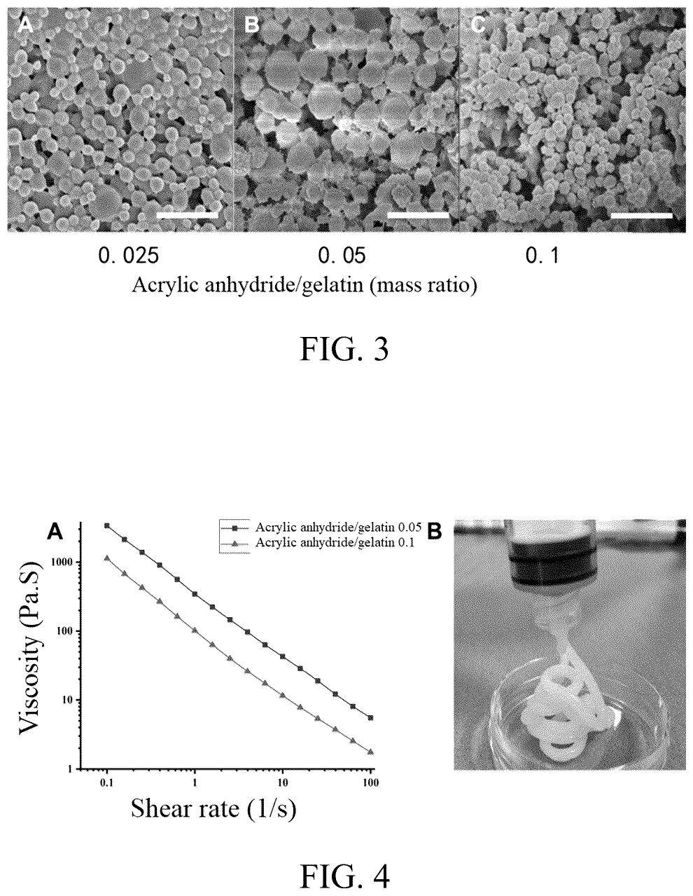

5 g of GelMA with different grafting degrees in Table 1 was re-dissolved in 100 mL of deionized water at 40° C. and pH thereof was adjusted to 2.5, followed by adding 300 mL of acetone within 30 min under rapid stirring conditions to slowly dehydrate and curl protein molecules to form nanospheres, and then 165 μL of crosslinking agent glutaraldehyde was added and stirred for 12 hours to obtain a granule suspension. The granule suspension was freeze-dried after adjusting pH thereof to 7 with sodium hydroxide, and GelMA nanoparticle powder was obtained. In the group with a grafting degree being 87%, the gelatin granules were not crosslinked by the crosslinking agent since a large number of amino groups were substituted; and crosslinked granules were obtained in other groups. Surface charge of granules obtained with different addition amount of methacrylic anhydride is shown in FIG. 1, and size and morphology of the granules thereof are shown in scanning electron microscopy images and transmission electron microscopy images of FIG. 2 and 3.

3. 0.08 g or 0.13 g of GelMA granular powder, 1 mL of deionized water and 0.005 g of photoinitiator irgcure2959 were repeatedly blown 10 times by means of a Luer adapter syringe to obtain injectable self-healing granular gel. A storage modulus G′ of the granular gel was obtained by using a time sweep mode of a rotational rheometer, and a self-healing efficiency was obtained by comparing the storage modulus of the granular gel before and after oscillatory shearing (with a strain of 0.1%-1000%). Self-healing efficiency is shown in Table 2 and shear-thinning properties are shown in FIG. 4, with a frequency of 1 Hz and a strain of 0.5%. It can be seen that the gelatin granular gel has excellent self-healing efficiency and shear-thinning mechanical characteristic, and has injectable and printable properties. The gelatin granular gel has mechanical strength higher than that of the gelatin granules without covalently crosslinked groups grafted in Comparative example 2.

TABLE 2
j

| | 0.025 | 0.025 | 0.05 | 0.05 | 0.1 | 0.1 |
|---|---|---|---|---|---|---|
| | (Methacrylic | (Methacrylic | (Methacrylic | (Methacrylic | (Methacrylic | (Methacrylic |
| | anhydride/gelatin) | anhydride/gelatin) | anhydride/gelatin) | anhydride/gelatin) | anhydride/gelatin) | anhydride/gelatin) |
| | 8% (w/v) | 13% (w/v) | 8% (w/v) | 13% (w/v) | 8% (w/v) | 13% (w/v) |
| Storage | 0.9 kPa | 3.8 kPa | 1.2 kPa | 4.1 kPa | 1.5 kPa | 4.7 kPa |
| modulus (G′) | | | | | | |
| Self-healing | 91% | 78% | 87% | 79% | 88% | 80% |
| efficiency | | | | | | |

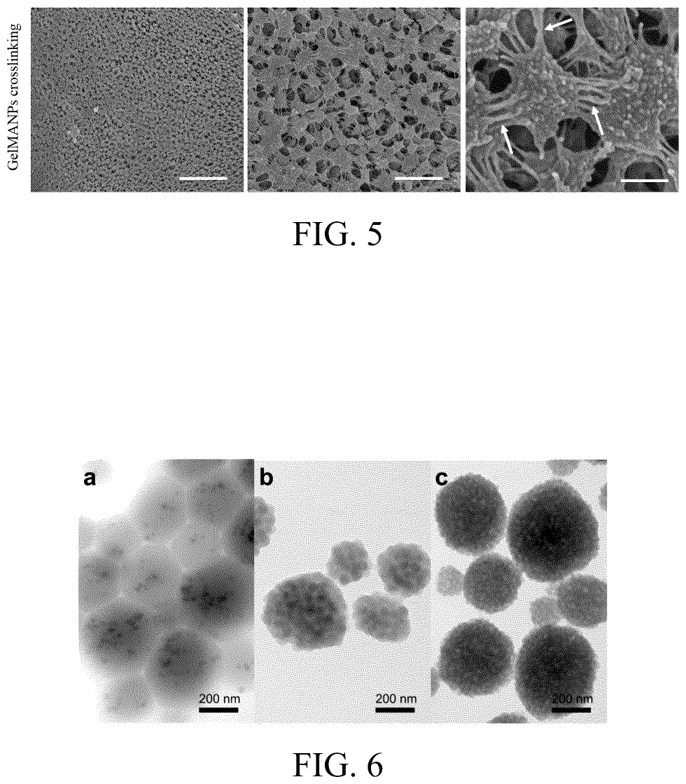

4. The injectable self-healing granular gel obtained in step 3 was irradiated for 30 S under an ultraviolet light intensity of 100 mW/m² to crosslink to obtain a high-strength GelMA colloidal gel. A scanning electron microscopy image of a structure of the high-strength GelMA colloidal hydrogel is shown in FIG. 5. A storage modulus G′ of the high-strength GelMA colloidal hydrogel obtained by using the time sweep mode of the rotational rheometer is shown in Table 3, with a frequency of 1 Hz and a strain of 0.5%. It can be seen that the strength of the gelatin granular hydrogel is enhanced after covalent crosslinking. Compared with a covalently crosslinked gelatin hydrogel in Comparative example 1, the covalently crosslinked gelatin granular hydrogel has higher mechanical strength (the storage modulus of the covalently crosslinked gelatin granular hydrogel with the same mass fraction and grafting degree of a covalent group is higher than that of a covalently crosslinked polymer hydrogel).

TABLE 3

| | 0.025 | 0.025 | 0.05 | 0.05 | 0.1 | 0.1 |
|---|---|---|---|---|---|---|
| | (Methacrylic | (Methacrylic | (Methacrylic | (Methacrylic | (Methacrylic | (Methacrylic |
| | anhydride/gelatin) | anhydride/gelatin) | anhydride/gelatin) | anhydride/gelatin) | anhydride/gelatin) | anhydride/gelatin) |
| | 8% (w/v) | 13% (w/v) | 8% (w/v) | 13% (w/v) | 8% (w/v) | 13% (w/v) |
| Storage | 17.3 kPa | 37.7 kPa | 22.6 kPa | 41.5 kPa | 26.1 kPa | 46.2 kPa |
| modulus (G′) | | | | | | |
| Self-healing | 31% | 26% | 33% | 24% | 29% | 21% |
| efficiency | | | | | | |

### Comparative Example 1

0.08 g and 0.13 g of GelMA freeze-dried sample prepared in step 1 in Embodiment 1 were respectively mixed with 1 mL of deionized water and 0.005 g of photoinitiator irgcure2959 at 40° C. to obtain two GelMA pre-polymerized solutions, followed by irradiating for 30 S under ultraviolet light intensity of 100 mW/m² to obtain two GelMA hydrogels. GelMA hydrogel is a representative gelatin polymer hydrogel. A storage modulus G′ of the hydrogel obtained by using the time sweep mode of the rotational rheometer is shown in Table 4, with a frequency of 1 Hz and a strain of 0.5%. By comparing the storage modulus, the strength of the covalently crosslinked GelMA polymer hydrogel is lower than that of the covalently crosslinked gelatin granular hydrogel under the same mass fraction (by data comparison of Table 4 and Table 3).

TABLE 4

| | 0.025 | 0.025 | 0.05 | 0.05 | 0.1 | 0.1 |
|---|---|---|---|---|---|---|
| | (Methacrylic | (Methacrylic | (Methacrylic | (Methacrylic | (Methacrylic | (Methacrylic |
| | anhydride/gelatin) | anhydride/gelatin) | anhydride/gelatin) | anhydride/gelatin) | anhydride/gelatin) | anhydride/gelatin) |
| | 8% (w/v) | 13% (w/v) | 8% (w/v) | 13% (w/v) | 8% (w/v) | 13% (w/v) |
| Storage | 3.7 kPa | 9.7 kPa | 6.2 kPa | 11.3 kPa | 7.2 kPa | 14.1 kPa |
| modulus (G′) | | | | | | |

### Comparative Example 2

5 g of gelatin powder was re-dissolved in 100 mL of deionized water at 40° C. and pH thereof was adjusted to 2.5, followed by adding 300 mL of acetone within 30 min under rapid stirring conditions to slowly dehydrate and curl protein molecules to form nanospheres, and then 165 μL of crosslinking agent glutaraldehyde was added and stirred for 12 hours to obtain a particle suspension. The particle suspension was freeze-dried after adjusting pH thereof to 7 with sodium hydroxide, and gelatin nanoparticle powder was obtained. 0.08 g or 0.13 g of gelatin nanoparticle powder respectively mixed with 1 mL of deionized water were repeatedly blown 10 times by means of a Luer adapter syringe to obtain two injectable self-healing granular gels. A storage modulus G′ of the granular gel was obtained by using a time sweep mode of a rotational rheometer, and a self-healing efficiency was obtained by comparing the storage modulus of the granular gel before and after oscillatory shearing (with a strain of 0.1%-1000%).

TABLE 5

| | 8% (w/v) | 13% (w/v) |
|---|---|---|
| Storage modulus | 0.8 kPa | 2.1 kPa |
| Self-healing property | 78% | 72% |

### Embodiment 2 (Different Granule Sizes)

1. Preparation of Methacrylate Grafted Gelatin Polymer

5 g of gelatin powder was dissolved in 100 mL of deionized water at 50° C. to obtain a gelatin solution. 0.25 g of methacrylic anhydride was added into the gelatin solution for reacting at a high temperature for two hours to obtain a reaction solution, and a nucleophilic substitution reaction occurred between the methacrylic anhydride and a free amino group on a protein molecular chain and equimolar methacrylic acid was produced. A pH value of the reaction solution wasis adjusted to 7 with hydrochloric acid, followed by adding acetone twice a volume of the reaction solution to destroy a hydration layer on a surface of a protein molecule, and polymer GelMA was precipitated out. The obtained methacrylate gelatin was repeatedly washed with deionized water and was freeze-dried to obtain a freeze-dried GelMA sample.

2. Preparation of GelMA Nanoparticles

The freeze-dried GelMA sample was re-dissolved in 100 mL of deionized water at 40° C., followed by heating to 45° C. and stirring for 30 minutes to obtain a clear and transparent GelMA solution. 300 mL of olive oil was added to a round-bottomed three-necked flask and heated to 45° C., followed by slowly adding 10 mL of GelMA aqueous solution with stirring in different manners for 15 min and keeping the temperature. The entire system was cooled to 4° C. by means of placing it in an ice bath while keeping stirring, after 30 minutes, 100 mL of acetone was added into the system with stirring for 15 minutes at a low temperature to obtain an emulsion. The cooling process can cause the gelatin droplets in the emulsion to produce a gel. Then the emulsion was filtered after adding another 15 mL of acetone, and the olive oil was removed by washing the emulsion with acetone continuously. Finally the filtrated product, i.e. GelMA microspheres, was collected. GelMA microspheres of 20 μm and 100 μm in size were respectively obtained by controlling a stirring speed.

3. 0.08 g or 0.13 g of GelMA microspheres, 1 mL of deionized water and 0.005 g of photoinitiator irgcure2959 were repeatedly blown 10 times by means of a Luer adapter syringe to obtain an injectable self-healing colloidal gel. A storage modulus G′ of the colloidal gel obtained by using a time sweep mode of a rotational rheometer and a self-healing efficiency are shown in Table 6, with a frequency of 1 Hz and a strain of 0.5%. Compared with nano-sized granules, micro-sized colloidal gels have lower mechanical strength than nano-sized colloidal gels without covalent crosslinking, but still have shear-thinning and self-healing properties.

TABLE 6

| | 5 μm | 5 μm | 50 μm | 50 μm |
|---|---|---|---|---|
| | 8% (w/v) | 13% (w/v) | 8% (w/v) | 13% (w/v) |
| Storage | 0.7 kPa | 2.2 kPa | 0.4 kPa | 1.8 kPa |
| modulus | | | | |
| (G′) | | | | |
| Self-healing | 73% | 65% | 72% | 65% |
| efficiency | | | | |

4. The injectable self-healing colloidal gel obtained in step 3 was irradiated for 30 S under an ultraviolet light intensity of 100 mW/m² to crosslink to obtain a gelatin granular hydrogel. A storage modulus G′ of the gelatin granular hydrogel obtained by using the time sweep mode of the rotational rheometer is shown in Table 7, with a frequency of 1 Hz and a strain of 0.5%. The strength of the micro-sized gelatin granular hydrogel after covalent crosslinking is obviously enhanced, and the mechanical strength thereof is reduced compared with that of the nano-sized gelatin granular hydrogel.

TABLE 7

| | 5 μm | 5 μm | 50 μm | 50 μm |
|---|---|---|---|---|
| | 8% (w/v) | 13% (w/v) | 8% (w/v) | 13% (w/v) |
| Storage | 8.4 kPa | 29.9 kPa | 7.1 kPa | 21.6 kPa |
| modulus | | | | |
| (G′) | | | | |
| Self-healing | 43% | 35% | 32% | 39% |
| efficiency | | | | |

### Embodiment 3 (Click Chemistry Manner)

1. Preparation of Gelatin Polymer by Click Chemistry

5 g of gelatin powder of type A was dissolved in 100 mL of deionized water at 50° C. to obtain a gelatin solution. 0.01 g of 1-(3-Dimethylaminopropyl)-3-ethylcarbodiimide hydrochloride (EDC)/N-hydroxysuccinimide (NHS) as a reaction catalyst and 0.1 g of azide imine or propynylamine were added to the gelatin solution to react for 2 hours to obtain a reaction solution, and the azide imine or propynylamine reacted nucleophilic substitution with free carboxyl groups on the gelatin chain to obtain an azide-terminated gelatin or an alkyne-terminated gelatin respectively. A pH value of the reaction solution was adjusted to 7 with hydrochloric acid, followed by adding acetone with a volume 2 times of the reaction solution to destroy a hydration layer on a surface of a gelatin molecule, and the azido group-terminated gelatin or the alkyne-terminated gelatin was precipitated out.

2. Preparation of Gelatin Nanoparticles

The precipitated azido group-terminated gelatin precipitate and alkyne-terminated gelatin were respectively re-dissolved in 100 mL of deionized water at 40° C. and the pH thereof were adjusted to 2.5, followed by adding 300 mL of acetone within 30 min under rapid stirring conditions to slowly dehydrate and curl protein molecules to form nanospheres, and then 165 μL of crosslinking agent glutaraldehyde was added and stirred for 12 hours to obtain two particle suspensions. pH of the two particle suspensions were respectively adjusted to 7 with sodium hydroxide followed by freeze-drying to respectively obtain azide gelatin granular powder and alkyne gelatin granular powder with size and surface charge shown in Table 8.

TABLE 8

| | Azide | Alkyne |
|---|---|---|
| | gelatin | gelatin |
| | granule | granule |
| Granule size | 416 nm | 467 nm |
| Surface charge | 11.9 mV | 7.9 mV |

3. 0.08 g or 0.13 g of gelatin granular powder obtained in step 2 and 1 mL of deionized water were repeatedly blown 10 times by means of a Luer adapter syringe to obtain an injectable self-healing granular gel. A storage modulus G′ of the granular gel obtained by using a time sweep mode of a rotational rheometer and a self-healing efficiency are shown in Table 9, with a frequency of 1 Hz and a strain of 0.5%.

TABLE 9

| | Azide/alkyne | Azide/alkyne |
|---|---|---|
| | gelatin | gelatin |
| | granule | granule |
| | 8% (w/v) | 13% (w/v) |
| Storage | 2.1 kPa | 5.8 kPa |
| modulus (G′) | | |
| Self-healing | 73.7% | 76.2% |
| efficiency | | |

4. The injectable self-healing granular gel obtained in step 3 was stood for 100 min and a high-strength covalently crosslinked granular gel was obtained after covalently crosslinking between surfaces of the granules. A storage modulus G′ of the granular gel obtained by using the time sweep mode of the rotational rheometer is shown in Table 10, with a frequency of 1 Hz and a strain of 0.5%.

TABLE 10

| | Azide/alkyne | Azide/alkyne |
|---|---|---|
| | gelatin | gelatin |
| | granule | granule |
| | 8% (w/v) | 13% (w/v) |
| Storage | 11.9 kPa | 28.9 kPa |
| modulus (G′) | | |
| Self-healing | 35% | 32% |
| efficiency | | |

### Embodiment 4 (Chemical Crosslinking Manner)

1. The GelMA nanoparticles prepared in Embodiment 1 were used.

2. 0.08 g or 0.13 g of GelMA nanoparticles, 1 mL of deionized water, 0.002 g of ammonium persulfate and N,N-tetramethylethylenediamine were repeatedly blown 10 times by means of a Luer adapter syringe to obtain an injectable self-healing granular gel. A storage modulus G′ of the granular gel obtained by using a time sweep mode of a rotational rheometer and a self-healing efficiency are shown in Table 11, with a frequency of 1 Hz and a strain of 0.5%.

TABLE 11

| | 0.025 | 0.025 | 0.05 | 0.05 | 0.1 | 0.1 |
|---|---|---|---|---|---|---|
| | (Methacrylic | (Methacrylic | (Methacrylic | (Methacrylic | (Methacrylic | (Methacrylic |
| | anhydride/gelatin) | anhydride/gelatin) | anhydride/gelatin) | anhydride/gelatin) | anhydride/gelatin) | anhydride/gelatin) |
| | 8% (w/v) | 13% (w/v) | 8% (w/v) | 13% (w/v) | 8% (w/v) | 13% (w/v) |
| Storage | 0.9 kPa | 3.8 kPa | 1.2 kPa | 4.1 kPa | 1.5 kPa | 4.7 kPa |
| modulus (G′) | | | | | | |
| Self-healing | 91% | 78% | 87% | 79% | 88% | 80% |
| efficiency | | | | | | |

3. The injectable self-healing granular gel obtained in step 2 was stood for 1 hour to obtain a high-strength GelMA granular gel. A storage modulus G′ of the granular gel obtained by using the time sweep mode of the rotational rheometer is shown in Table 1, with a frequency of 1 Hz and a strain of 0.5%. The strength of the crosslinked gel obtained by using the chemical crosslinking agent is similar to that of the colloidal gel covalently crosslinked by ultraviolet light in Embodiment 1.

TABLE 12

| | 0.025 | 0.025 | 0.05 | 0.05 | 0.1 | 0.1 |
|---|---|---|---|---|---|---|
| | (Methacrylic | (Methacrylic | (Methacrylic | (Methacrylic | (Methacrylic | (Methacrylic |
| | anhydride/gelatin) | anhydride/gelatin) | anhydride/gelatin) | anhydride/gelatin) | anhydride/gelatin) | anhydride/gelatin) |
| | 8% (w/v) | 13% (w/v) | 8% (w/v) | 13% (w/v) | 8% (w/v) | 13% (w/v) |
| Storage | 18.1 kPa | 34.7 kPa | 21.6 kPa | 40.5 kPa | 22.9 kPa | 43.1 kPa |
| modulus (G′) | | | | | | |
| Self-healing | 30% | 29% | 33% | 24% | 29% | 21% |
| efficiency | | | | | | |

### Embodiment 5 (Silicon Dioxide with Methacrylate Gelatin)

1. 5 g of gelatin powder of type A was dissolved in 100 mL of deionized water at 50° C. to obtain a gelatin solution, 0.5 g of methacrylic anhydride was added into the gelatin solution for reacting at a high temperature for two hours to obtain a reaction solution, a nucleophilic substitution reaction occurred between the methacrylic anhydride and a free amino group on a protein molecular chain occurrs, and equimolar methacrylic acid was also produced. pH of the reaction solution was adjusted to 7 with hydrochloric acid, followed by adding acetone twice a volume of the reaction solution is added to destroy a hydration layer on a surface of a protein molecule, and the GelMA was precipitated out. The obtained GelMA was repeatedly washed with deionized water to remove impurities followed by freeze-drying for standby.

2. The GelMA prepared above was re-dissolved in 100 mL of silicon dioxide granular suspension with concentration of 1 mg/ml, 10 mg/ml and 20 mg/ml respectively (prepared through a Stober method, with a granule size of 50 nm) at 40° C. and, pH thereof was adjusted to 2.5, followed by adding 300 mL of acetone within 30 min under rapid stirring conditions to slowly dehydrate and curl protein molecules to form nanospheres, and an obtained solution was milky white. Then 165 μL of glutaraldehyde was added with stirring for 12 hours to crosslink the gelatin nanoparticles, and then crosslinking was terminated by adding the same volume of 100 mM glycine. After 2 h, an obtained suspension was centrifuged followed by washing a precipitate 3 times, and then the precipitate was re-dispersed into deionized water to obtain a silicon dioxide/methacrylate gelatin core-shell nanoparticle suspension.

3. The size and morphology of the silicon dioxide nanoparticles and the silicon dioxide/methacrylate gelatin core-shell nanoparticles were observed by transmission electron microscopy. As shown in FIG. 6 panel a, the size of the silicon dioxide nanoparticles is about 50 nm, and the size of the silicon dioxide/methacrylate gelatin core-shell nanoparticles is about 300 nm, and it can be clearly observed by transmission electron microscopy that a large amount of dark black silicon dioxides is wrapped inside the light gray GelMA granules to form a core-shell structure (FIG. 6).

4. 0.1 g or 0.2 g of the aforementioned core-shell nanoparticles, 1 mL of deionized water and 0.005 g of photoinitiator irgcure2959 were repeatedly blown 10 times by means of a Luer adapter syringe to obtain an inorganic-reinforced injectable and self-healing colloidal gel with a core-shell structure. A storage modulus G′ of the colloidal gel was obtained by using a time sweep mode of a rotational rheometer, and a self-healing efficiency was obtained by comparing the storage modulus of the colloidal gel before and after oscillatory shearing (with a strain of 0.1%-1000%), shown in Table 13. It can be seen that the colloidal gel with a core-shell structure has excellent self-healing properties.

TABLE 13

| | 0.025 | 0.025 | 0.1 | 0.1 |
|---|---|---|---|---|
| | (Methacrylic- | (Methacrylic- | (Methacrylic- | (Methacrylic- |
| | anhydride/ | anhydride/ | anhydride/t | anhydride/ |
| | gelatin) | gelatin) | gelain) | gelatin) |
| | 8% (w/v) | 13% (w/v) | 8% (w/v) | 13% (w/v) |
| Storage | 5.0 kPa | 31.8 kPa | 4.8 kPa | 30.5 kPa |
| modulus | | | | |
| (G′) | | | | |
| Self-healing | 91% | 78% | 92% | 76% |
| efficiency | | | | |

5. The injectable and self-healing colloidal gel obtained in step 4 was irradiated for 30 S under an ultraviolet light intensity of 100 mW/m³ to crosslink to obtain a high-strength colloidal hydrogel with a core-shell structure. A storage modulus G′ of the colloidal hydrogel with a core-shell structure obtained by using the time sweep mode of the rotational rheometer is shown in Table 14, with a frequency of 1 Hz and a strain of 0.5%.

TABLE 14

| | 0.025 | 0.025 | 0.1 | 0.1 |
|---|---|---|---|---|
| | (Methacrylic- | (Methacrylic- | (Methacrylic- | (Methacrylic- |
| | anhydride/ | anhydride/ | anhydride/ | anhydride/ |
| | gelatin) | gelatin) | gelatin) | gelatin) |
| | 8% (w/v) | 13% (w/v) | 8% (w/v) | 13% (w/v) |
| Storage | 21.1 kPa | 51.4 kPa | 33.7 kPa | 71.3 kPa |
| modulus | | | | |
| (G′) | | | | |
| Self-healing | 31% | 22% | 20% | 16% |
| efficiency | | | | |

### Embodiment 6 (Compounding Ferroferric Oxide With Gelatin)

1. GelMA nanoparticle powder with a ratio of Gelatin acrylate to gelatin of 0.1, prepared in Embodiment 1, was dissolved in 100 mL of 5 mg/mL ferroferric oxide granular suspension (purchased from Sigma-Aldrich Technologies) at 40° C., and pH thereof was adjusted to 10.5, followed by adding 330 mL of acetone within 30 min under rapid stirring conditions to slowly dehydrate and curl protein molecules to form nanospheres, and an obtained solution was milky white. Then 165 μL of glutaraldehyde was added to the above obtained solution with stirring for 12 hours to crosslink the nanospheres, and crosslinking was terminated by adding the same volume of 100 mM glycine. After 2 hours, an obtained suspension was centrifuged followed by washing a precipitate thereof 3 times, and then the precipitate were re-dispersed into deionized water to obtain a ferroferric oxide/methacrylate gelatin core-shell nanoparticle suspension.

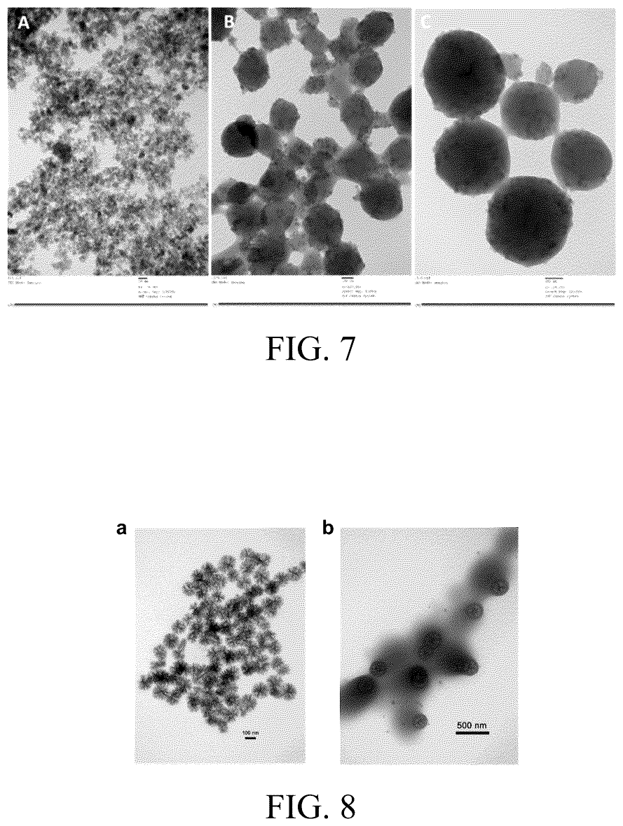

2. The size and morphology of the ferroferric oxide granules and the ferroferric oxide/methacrylate gelatin core-shell nanoparticles were observed by transmission electron microscopy. As shown in FIG. 7 panel A, the size of the ferroferric oxide granules is about 50 nm, and the size of the ferroferric oxide/methacrylate gelatin core-shell nanoparticles is about 300 nm, and it can be clearly observed by transmission electron microscopy that a large amount of dark black ferroferric oxide is wrapped inside light gray GelMA granules to form a core-shell structure (FIG. 7 panels B and C).

3. 0.2 g of core-shell nanoparticles prepared above, 1 mL of deionized water and 0.005 g of photoinitiator irgcure2959 were repeatedly blown 10 times by means of a Luer adapter syringe to obtain an injectable self-healing colloidal gel. A storage modulus G′ of the injectable self-healing colloidal gel obtained by using a time sweep mode of a rotational rheometer and a self-healing efficiency are shown in Table 15, with a frequency of 1 Hz and a strain of 0.5%.

TABLE 15

| Storage modulus | 29.4 kPa |
|---|---|
| (G′) | |
| Self-healing efficiency | 88% |

4. The injectable self-healing colloidal gel obtained in step 3 was irradiated for 30 S under an ultraviolet light intensity of 100 mW/m² to crosslink to obtain a high-strength colloidal hydrogel with a core-shell structure. A storage modulus G′ of the high-strength colloidal hydrogel with a core-shell structure obtained by using the time sweep mode of the rotational rheometer is shown in Table 16, with a frequency of 1 Hz and a strain of 0.5%.

TABLE 16

| Storage modulus | 51.4 kPa |
|---|---|
| (G′) | |
| Self-healing efficiency | 25% |

### Embodiment 7 (Mesoporous Bioglass)

1. Bioglass was prepared through an emulsion method. First, 1.6 g of Cetyltrimethyl

Ammonium Bromide (CTAB) was added to 100 mL of deionized water with stirring until a clear solution was obtained, then pH thereof was adjusted to 7.2, followed by adding 6.25 mL of tetraethyl orthosilicate (TEOS), 4.3 g of calcium nitrate and 2.1 mL of triethyl phosphate with stirring at 600 rpm for 12 h to obtain a product. The product was sintered at 600° C. for 3 h after centrifuging and washing to obtain the mesoporous bioglass granules.

2. The GelMA nanoparticle powder prepared in Embodiment 1 was dissolved in 100 mL of 5 mg/mL mesoporous bioglass granular suspension at 40° C. and pH thereof was adjusted to 2.5, followed by adding 330 mL of acetone within 30 min under rapid stirring conditions to slowly dehydrate and curl protein molecules to form nanospheres, and an obtained solution was milky white. Then 165 μL of glutaraldehyde was added with stirring for 12 h to crosslink the nanospheres, and crosslinking was terminated by adding the same volume of 100 mM glycine. After 2 h, an obtained suspension was centrifuged followed by washing a precipitate thereof 3 times, and then the precipitate were re-dispersed into deionized water to obtain a mesoporous bioglass/GelMA core-shell nanoparticle suspension.

3. The size and morphology of the above mesoporous bioglass granules and the mesoporous bioglass/GelMA core-shell granules were observed by transmission electron microscopy. As shown in FIG. 8 panel a, the size of the mesoporous bioglass granules is about 100 nm, and the size of the mesoporous bioglass/GelMA core-shell granules is about 300 nm, and it can be clearly observed by transmission electron microscopy that a large amount of dark black mesoporous bioglass is wrapped inside the light gray GelMA granules to form a core-shell structure (FIG. 8 panel b).

4. 0.2 g of the core-shell granules prepared above, 1 mL of deionized water and 0.005 g of photoinitiator irgcure2959 were repeatedly blown 10 times by means of a Luer adapter syringe to obtain an injectable self-healing colloidal gel. A storage modulus G′ of the injectable self-healing colloidal gel obtained by using a time sweep mode of a rotational rheometer and a self-healing efficiency are shown in Table 17, with a frequency of 1 Hz and a strain of 0.5%. Compared with nano-sized granules, micron-sized gelatin microspheres have slightly reduced self-healing efficiency, but have shear-thinning and self-healing properties.

TABLE 17

| Storage modulus | 27.7 kPa |
|---|---|
| (G′) | |
| Self-healing efficiency | 81% |

5. The injectable self-healing colloidal gel obtained in step 4 was irradiated for 30 S under an ultraviolet light intensity of 100 mW/m² to crosslink to obtain a high-strength colloidal hydrogel with a core-shell structure. A storage modulus G′ of the high-strength colloidal hydrogel obtained by using the time sweep mode of the rotational rheometer is shown in Table 18, with a frequency of 1 Hz and a strain of 0.5%.

TABLE 18

| Storage modulus | 55.4 kPa |
|---|---|
| (G′) | |
| Self-healing efficiency | 23% |

### Embodiment 8 (Hydroxyapatite)

1. The GelMA nanoparticle powder prepared in Embodiment 1 was dissolved in 100 mL of 5 mg/mL hydroxyapatite (purchased from Sigma-Aldrich Technologies, with a granule size of 150 nm) suspension at 40° C. and pH thereof was adjusted to 2.5, followed by adding 330 mL of acetone within 30 min under rapid stirring conditions to slowly dehydrate and curl protein molecules to form nanospheres, and an obtained solution was milky white. Tand then 165 μL of glutaraldehyde was added with stirring for 12 h to crosslink the nanopheres, and crosslinking was terminated by adding the same volume of 100 mM glycine. After 2 h, an obtained suspension was centrifuged followed by washing a precipitate 3 times, and then the precipitate were re-dispersed into deionized water to obtain a hydroxyapatite/GelMA core-shell nanoparticle suspension.

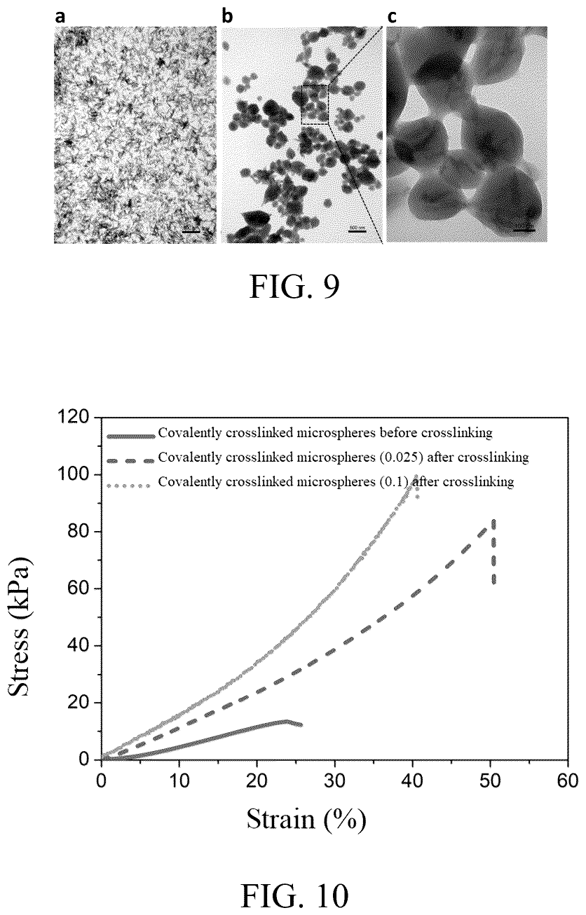

2. The size and morphology of the hydroxyapatite granules and the hydroxyapatite/GelMA core-shell nanoparticles were observed by transmission electron microscopy. As shown in FIG. 9 panel a, hydroxyapatite is of acicular shape, the size of the hydroxyapatite/GelMA core-shell nanoparticles is about 500 nm, and it can be clearly observed by transmission electron microscopy that a large amount of dark black hydroxyapatite is wrapped inside the light gray GelMA granules to form a core-shell structure (FIG. 9 panels b and c).

3. 0.2 g of the core-shell nanoparticles prepared above, 1 mL of deionized water and 0.005 g of photoinitiator irgcure2959 were repeatedly blown 10 times by means of a Luer adapter syringe to obtain an injectable self-healing colloidal gel with a core-shell structure. A storage modulus G′ of the injectable self-healing colloidal gel was obtained by using a time sweep mode of a rotational rheometer, and self-healing efficiency is shown in Table 19, with a frequency of 1 Hz and a strain of 0.5%.

TABLE 19

| Storage modulus | 23.1 kPa |
|---|---|
| (G′) | |
| Self-healing efficiency | 89% |

4. The injectable self-healing colloidal gel with a core-shell structure obtained in step 3 was irradiated for 30 S under an ultraviolet light intensity of 100 mW/m² to crosslink to obtain a high-strength colloidal hydrogel with a core-shell structure. A storage modulus G′ of the high-strength colloidal hydrogel obtained by using the time sweep mode of the rotational rheometer is shown in Table 20, with a frequency of 1 Hz and a strain of 0.5%.

TABLE 20

| Storage modulus | 51.2 kPa |
|---|---|
| (G′) | |
| Self-healing efficiency | 20% |

### Embodiment 9

Compression tests were performed on the covalently crosslinked granular gels prepared in Embodiments 1-4. The covalently crosslinked granular gels were made into cylinders (with a diameter of 6.4 mm and a height of 6 mm) for the compression test. With the colloidal gel prepared in Embodiment 1 as an example, the compression test was carried out at a loading rate of 0.0002 mm/s. An elastic modulus of a sample was computed from an average slope of an initial portion (0 to 10% strain) of a stress-strain curve of the sample. A compressive stress-strain curve of the sample is shown in FIG. 10, it can be seen that mechanical strength of the gel is significantly increased after covalent crosslinking, and the higher the grafting degree of a covalent group, the greater the mechanical strength of the gel.

### Comparative Example 3

Compression test was performed on the granular gel prepared in Comparative example 2. The granular gel was made into a cylinder (with a diameter of 6.4 mm and a height of 6 mm) for the compression test. The compression test was carried out at a loading rate of 0.0002 mm/s. An elastic modulus of a sample was computed from an average slope of an initial portion (0 to 10% strain) of a stress-strain curve of the sample. A stress-strain curve of the sample is shown in FIG. 10. Compared with the covalently crosslinkable granular gel prepared in Embodiment 5, the non-covalently crosslinkable granular gel has low mechanical strength and poor compression properties.

### Embodiment 10

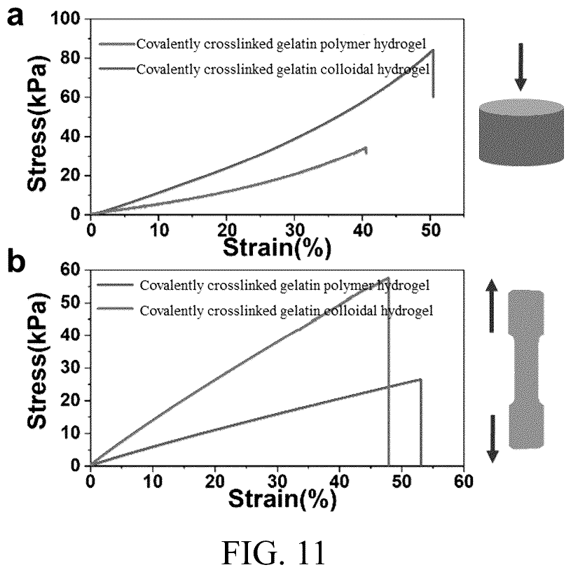

Compression and tensile tests were performed on the covalently crosslinked granular gels prepared in Embodiments 1-4. The covalently crosslinked granular gels were made into cylinders (with a diameter of 8 mm and a height of 6 mm). With the colloidal gel having a mass fraction of 13% and a ratio of methacrylic anhydride/gelatin of 0.05 prepared in Embodiment 1 as an example, the tensile and compression tests were carried out at a loading rate of 10 mm/min. An elastic modulus of a sample was computed from an average slope of an initial portion (0 to 10% strain) of a stress-strain curve of the sample. A stress-strain curve of the sample is shown in FIG. 11.

### Comparative Example 4

Compression and tensile tests were performed on the covalently crosslinked hydrogel prepared in Comparative example 1. The covalently crosslinked hydrogel was made into a cylinder (with a diameter of 8 mm and a height of 6 mm). With the covalently crosslinked gelatin hydrogel having a mass fraction of 13% and a ratio methacrylic anhydride/gelatin of 0.05 as an example, the tensile and compression tests were carried out at a loading rate of 10 mm/min. An elastic modulus of a sample was computed from an average slope of an initial portion (0 to 10% strain) of a stress-strain curve of the sample. A stress-strain curve of the sample is shown in FIG. 11. Mechanical strength of the covalently crosslinked hydrogel prepared in Comparative example 1 is lower than that of the covalently crosslinked granular gel prepared in Embodiment 6.

### Embodiment 11

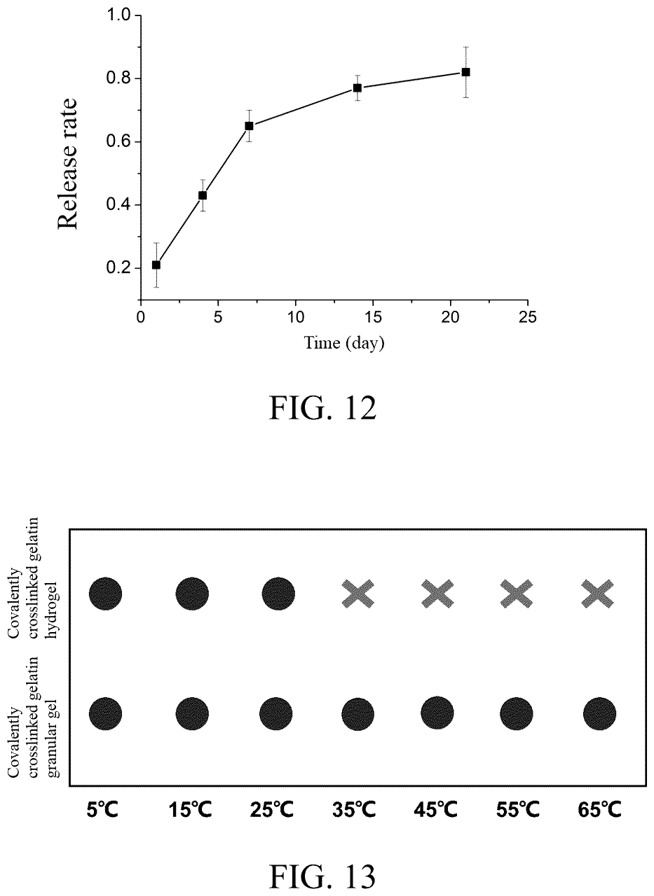

The covalently crosslinked colloidal gels prepared in Embodiments 1-4 were used for test. As an example, the covalently crosslinkable granular gel prepared in Embodiment 1 was placed on a shaker (30 rpm/min) at an ambient temperature of 37° C. to simulate a dynamic environment in vivo. 1 mL of phosphate buffer saline (PBS) supernatant was absorbed at 1st d, 3rd d, 7th d, 14th d and 21th d separately, and then an equal amount of fresh PBS solution was added. A content of a vascular endothelial growth factor (VEGF) is measured at each time point by using an enzyme-linked immunosorbent assay (ELISA) kit with three samples per group. As shown in FIG. 12, by means of ELISA detection, it can be found that the VEGF in the covalently crosslinkable colloidal gel is slowly released at a relatively constant concentration, and such uniform release can still be detected on the 21th day, indicating that the covalently crosslinkable colloidal gel has a drug sustained release property and can be used as a carrier of bioactive sub stances.

### Embodiment 12

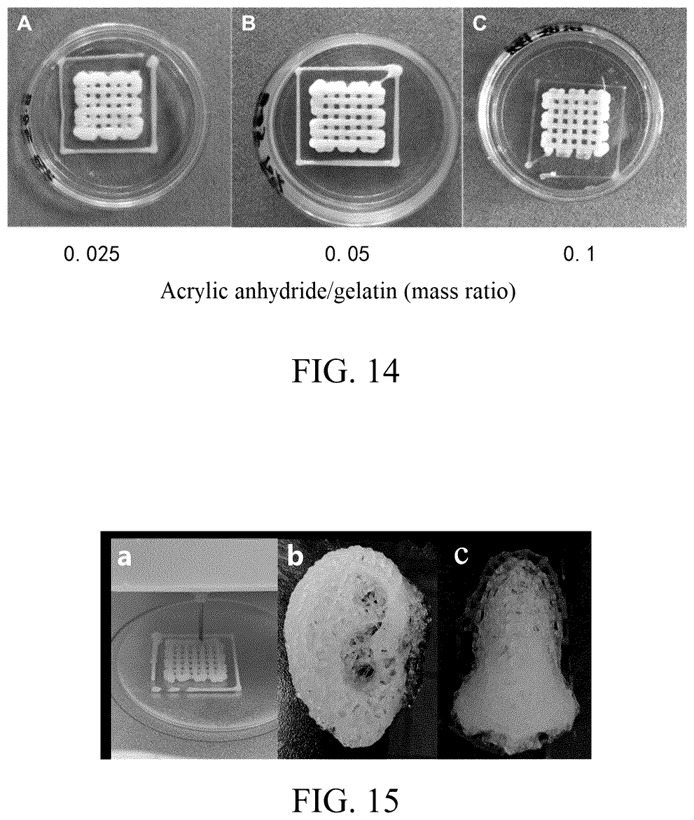

The granular gels prepared in Embodiments 1-4 were used for test. An uncovalently crosslinked granular gel was loaded into a syringe, and was printed by using a three dimensional (3D) bioprinter through a needle with a caliber of G16-23. The material was printed layer by layer according to a route designed by an established program to obtain a 3D bioprinted scaffold with a fine structure. With the granular gel prepared in Embodiment 1 as an example, 1 mL of culture medium solution, 0.13 g of methacrylate gelatin granules and 0.005 g of Lap ultraviolet initiator were blended to obtain a printable methacrylate gelatin granular gel, which was continuously extruded to form a filiform shape through a 3D bioprinter at different temperatures to evaluate printability of the granular gel in different environments. The printability of the granular gel at different temperatures is shown in FIG. 13, showing that the granular gel can be stably printed at 5° C. to 65° C. The granular gel was further printed layer by layer through a 3D bioprinter according to a route designed by an established program to obtain a scaffold, and the scaffold was irradiated with an ultraviolet lamp for 30 seconds for covalent crosslinking between granules, so as to obtain a granular gel scaffold with a stable mechanical structure. A process of 3D bioprinting and a bioprinted structure are shown in FIGS. 14 and 15. It can be seen from the figures that the scaffold structure is clear and complete, indicating that the covalently crosslinkable granular gel has an excellent printing property.

### Embodiment 13

With primary mesenchymal stem cells of a mouse as an example, proliferation culture was carried out (a dulbecco's modified eagle medium (DMEM) containing 10% of fetal bovine serum (FBS, Gibco)) at 37° C. with relative humidity of 95% and carbon dioxide of 5%, and the cell culture medium was replaced every two days. Before cultivation, the cells were buffered with phosphate buffered saline (PBS) and detached in a trypsin/ethylene diamine tetraacetic acid (EDTA) solution (0.25% trypsin/0.02% EDTA) for 5 minutes, and then suspended in the culture medium for standby. The colloidal gels prepared in Embodiments 1-6 were used as two-dimensional culture substrates, and the cell suspension was dropped directly onto a surface of the scaffold prepared in Embodiment 10, inoculated at a cell concentration of 5000 cell/cm², and then added into the culture medium to perform proliferation culture after standing for 1 h.

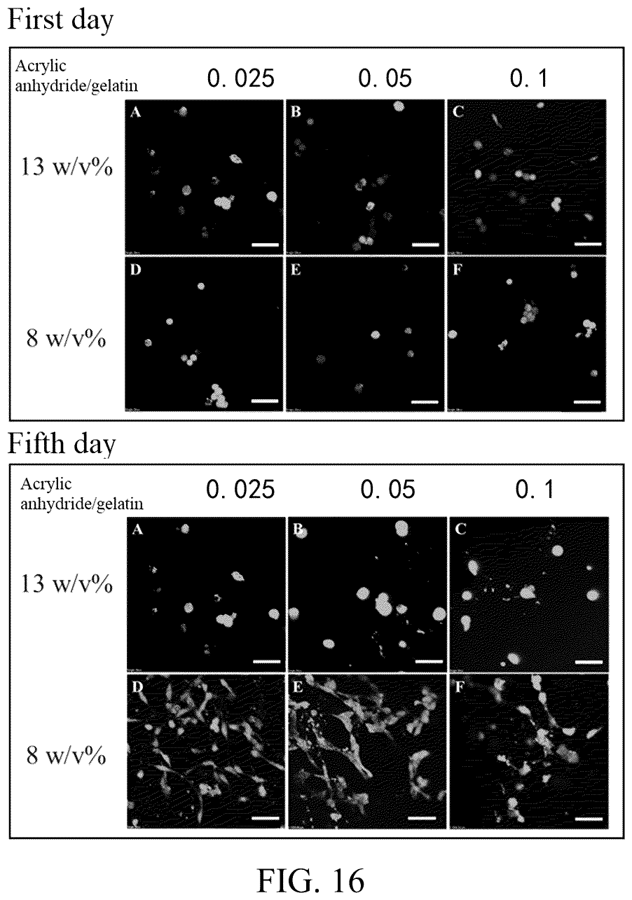

Cytotoxicity of the gel material was observed by means of Live/Dead assay. 2 mM calcein (green fluorescent labeled live cells) and 4 mM ethidium homodimer (red fluorescent labeled dead cells) were added to a cultured product at a room temperature, and confocal laser scanning microscopy was used to observe. With the granular gel prepared in Embodiment 1 as an example, results are shown in FIG. 16, where green fluorescence represents living cells and red fluorescence represents dead cells. It can be seen that the 3T3 cells cultured on the granular hydrogel for a long time are all living cells, indicating that the granular hydrogel has excellent biocompatibility.

### Embodiment 14

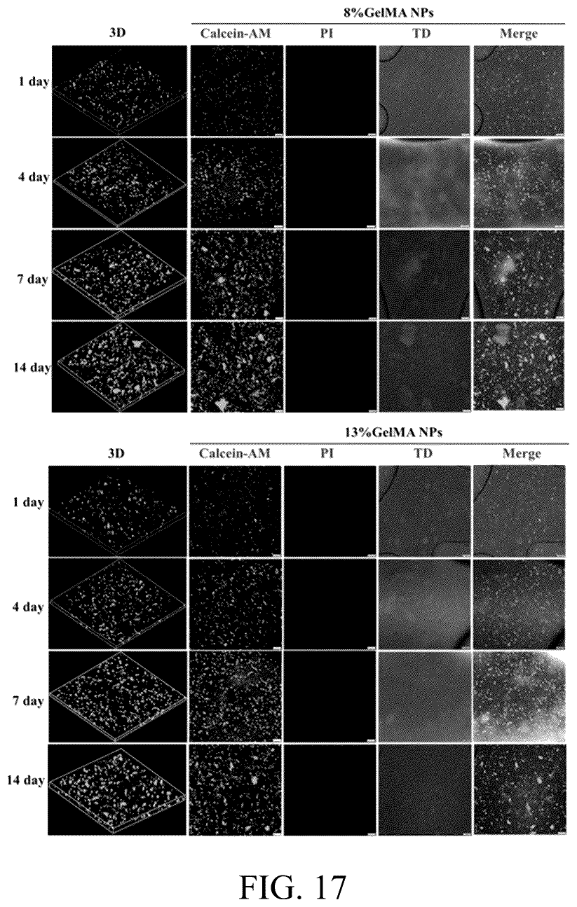

With primary mesenchymal stem cells of a mouse as an example, proliferation culture (a dulbecco's modified eagle medium (DMEM) containing 10% of fetal bovine serum (FBS, Gibco)) was carried out at 37° C. with relative humidity of 95% and carbon dioxide of 5%, and the cell culture medium was replaced every two days. Before cultivation, the cells were buffered with phosphate buffered saline (PBS) and detached in a trypsin/ethylene diamine tetraacetic acid (EDTA) solution (0.25% trypsin/0.02% EDTA) for 5 minutes, and then suspended in the culture medium for standby. 1 mL of cell suspension, 0.08 g or 0.13 g of methacrylate gelatin granules and 0.005 g of Lap ultraviolet initiator were blended to obtain a printable methacrylate gelatin granular gel. The obtained gelatin granular gel was further printed layer by layer through a 3D bioprinter according to a route designed by an established program to obtain a scaffold, and the scaffold was further irradiated with an ultraviolet lamp for 20 seconds for covalent crosslinking between granules, so as to obtain a granular gel scaffold with a stable mechanical structure. Then the scaffold was added to the culture medium for cultivation and observation. A cell survival rate in the gel material was investigated by using a cell counting kit (CCK) to detect cell viability. The cell survival rate in a printing process is shown in Table 21. It can be seen that the cell survival rate of the colloidal gel with a mass fraction of 8% is higher than that with a mass fraction of 13%, indicating that the gel with a lower mass fraction in an injection process can reduce a shear force on the cells during the injection process and thus improve the cell viability. The cells in the gel material were observed by means of Live/Dead assay. 2 mM calcein (green fluorescent labeled live cells) and 4 mM ethidium homodimer (red fluorescent labeled dead cells) were added to a cultured product at a room temperature, and confocal laser scanning microscopy was used to observe. Results are shown in FIG. 17, where green fluorescence represents living cells and red fluorescence represents dead cells. It can be seen that the mesenchymal stem cells cultured on the hydrogel for a long time are all living cells, indicating that the covalently crosslinked gelatin granules have excellent biocompatibility and are ideal cell carriers.

TABLE 21

| Mass fraction | Cell survival rate |
|---|---|
| 8% | 93% |
| 13% | 86% |

### Comparative Example 5

With primary mesenchymal stem cells of a mouse as an example, proliferation culture was carried out (a dulbecco's modified eagle medium (DMEM) containing 10% of fetal bovine serum (FBS, Gibco)) at 37° C. with relative humidity of 95% and carbon dioxide of 5%, and the cell culture medium was replaced every two days. Before cultivation, the cells were buffered with phosphate buffered saline (PBS) and detached in a trypsin/ethylene diamine tetraacetic acid (EDTA) solution (0.25% trypsin/0.02% EDTA) for 5 minutes, and then suspended in the culture medium for standby. 1 mL of cell suspension, 0.08 g or 0.13 g of methacrylate gelatin and 0.005 g of Lap ultraviolet initiator were blended to obtain a printable methacrylate gelatin colloidal gel. The obtained gelatin colloidal gel was printed layer by layer through a 3D bioprinter according to a route designed by an established program in a low temperature environment of 4° C. to obtain a scaffold, and the scaffold was further irradiated with an ultraviolet lamp for 20 seconds for covalent crosslinking between granules, so as to obtain a hydrogel scaffold. Then the scaffold was added to the culture medium for cultivation and observation. A cell survival rate in the gel material was investigated by using a cell counting kit (CCK) to detect cell viability. The cell survival rate in a printing process is shown in Table 22.

TABLE 22

| Mass fraction | Cell survival rate |
|---|---|
| 8% | 91% |
| 13% | 89% |

### Comparative Example 6

With primary mesenchymal stem cells of a mouse as an example, proliferation culture was carried out (a dulbecco's modified eagle medium (DMEM) containing 10% of fetal bovine serum (FBS, Gibco)) at 37° C. with relative humidity of 95% and carbon dioxide of 5%, and the cell culture medium was replaced every two days. Before cultivation, the cells were buffered with phosphate buffered saline (PBS) and detached in a trypsin/ethylene diamine tetraacetic acid (EDTA) solution (0.25% trypsin/0.02% EDTA) for 5 minutes, and then suspended in the culture medium for standby. 1 mL of cell suspension and 0.08 g of gelatin granular powder were blended to obtain a printable gelatin granular gel. The obtained printable gelatin granular gel was printed layer by layer through a 3D bioprinter according to a route designed by an established program to obtain a scaffold. Since the printed scaffold contains no covalently crosslinked groups, it is found that physical interaction of a non-covalently crosslinked gel cannot maintain integrity thereof at a low mass fraction when the printed scaffold was directly added to the culture medium for culture, so that the printed scaffold was easy to disperse in the culture medium.

### Embodiment 15

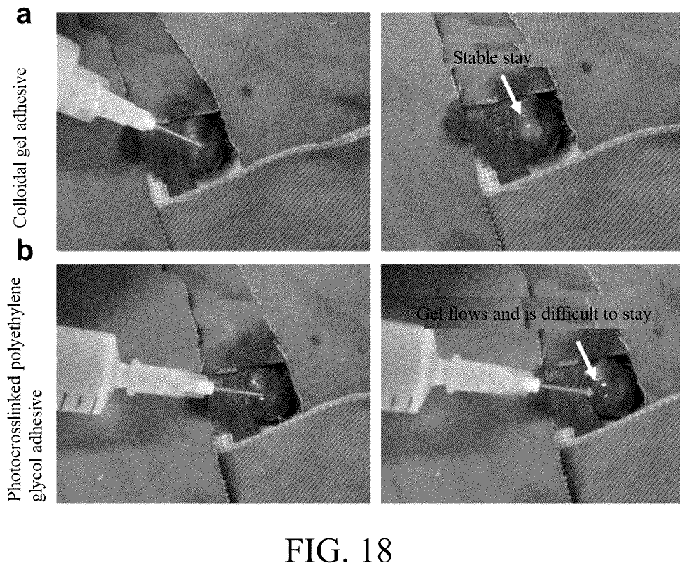

The covalently crosslinkable gelatin granular gels prepared in Embodiments 1-4 were used for test, and Sprague-Dawley (SD) rat heart injury was used as an animal experimental model. With the granular gel prepared in Embodiment 1 as an example, 1 mL of culture medium solution, 0.13 g of methacrylate gelatin granules and 0.005 g of Lap ultraviolet initiator were blended to obtain a granular gel adhesive. The granular gel adhesive was injected on a surface of the beating heart, and the granular gel adhesive can be easily injected onto the tissue surface and stably stays thereon without flowing. An experimental process is shown in FIG. 18 panel a.

### Comparative Example 7

A pre-polymerized solution of polyethylene glycol diacrylate with a mass fraction of 20% and a molecular weight of 8 kDa was used as a commercial available photo-crosslinked liquid tissue adhesive for control. The pre-polymerized solution quickly flowed down the surface of beating heart after being injected onto a surface of a beating heart, unable to stay at a designated site. An experimental process is shown in FIG. 18 panel a.

### Embodiment 16

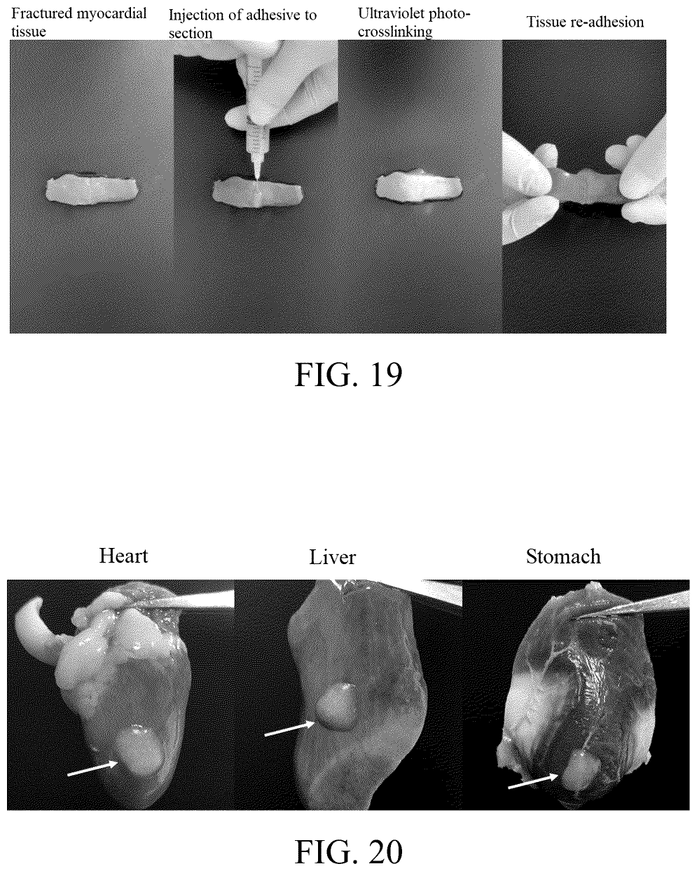

The covalently crosslinkable gelatin granular gels prepared in Embodiments 1-4 were used for test. With the granular gel prepared in Embodiment 1 as an example, 1 mL of culture medium solution, 0.13 g of methacrylate gelatin colloidal granules and 0.005 g of Lap ultraviolet initiator were blended to obtain a methacrylate gelatin granular gel adhesive. After fractured myocardial tissue was re-contacted, as shown in FIG. 19, the granular gel adhesive was injected at the fracture site followed by irradiating with ultraviolet light for 20 sec, then the fractured tissue was pulled up to re-adhere together and can remain stable. The granular gel adhesive as further injected onto surfaces of ex vivo heart, liver and stomach and irradiated with ultraviolet light for 30 sec to covalent crosslink the granules and form stable adhesion with the tissue. The covalently crosslinkable granular gel formed stable adhesion with the tissue, as shown in FIG. 20.

### Embodiment 17

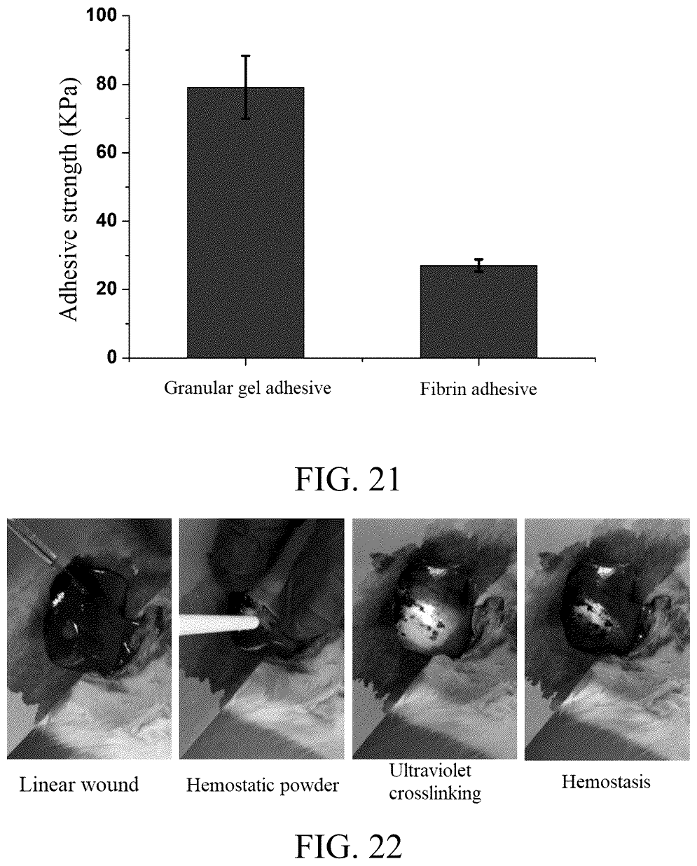

The covalently crosslinkable gelatin granular gels prepared in Embodiments 1-4 were used for test. With the granular gel prepared in Embodiment 1 as an example, 1 mL of deionized water, 0.13 g of methacrylate gelatin colloidal granules and 0.005 g of Lap ultraviolet initiator were blended to obtain a gelatin granular gel. The gelatin granular gel was lap-bonded between surfaces of two pieces of glass (being a 5.0 cm*2.0 cm rectangle), where an overlapping area was a 1.5 cm*2.0 cm rectangle. Then the overlapping area was irradiated with ultraviolet light of 50 mW/cm² for 20 sec. After standing for 10 min, shear peeling was performed on the lap-bonded glasses through a tensile tester with a 50 N load cell (with a peel rate of 10 mm/min), and a stress-strain curve during peeling was obtained. Adhesive strength was defined by using the maximum stress point of the curve. The maximum adhesive strength is shown in FIG. 21.

### Comparative Example 8

A commercial available fibrin tissue adhesive was lap-bonded between surfaces of two pieces of glass (being 5.0 cm*2.0 cm rectangle), where an overlapping area was a 1.5 cm*2.0 cm rectangle. After standing for 10 min, shear peeling was performed on the lap-bonded glasses through a tensile tester with a 50 N load cell (with a peel rate of 10 mm/min), and a stress-strain curve during peeling was obtained. Adhesive strength was defined by using a maximum stress point of a curve. The maximum adhesive strength is shown in FIG. 21.

### Embodiment 18

The covalently crosslinkable gelatin granular powder prepared in Embodiments 1-4 was loaded in a powder spray bottle. With a SD rat liver wound defect as an animal experimental model, a liver was cut with a scalpel to create a bleeding wound to form a defect area with a linear length of 10 mm. With the covalently crosslinkable gelatin granular powder prepared in Embodiment 1 as an example, 1 g of the granular powder and 0.05 g of a photo-crosslinking agent Irgacure2959 were blended and sprayed on a bleeding wound surface. The blended powder was quickly adsorbed on a tissue surface of the bleeding wound surface. After wound bleeding stops, the blended powder had been transformed into a granular gel due to the absorption of blood, followed by irradiating the granular gel in the wound area with ultraviolet light for 10 s to initiate covalent crosslinking on granular surfaces, so that the granular gel and the tissue surface form mechanical interlocking due to a covalent crosslinking interface, thus achieving stable adhesion between the granular gel and the tissue. Moreover, due to the covalent crosslinking between the granules, the strength of the granular gel was significantly increased, and the granular gel was not easy to cause entire adhesion failure caused by break of its own structure in the adhesion process. As shown in FIG. 22, rapid hemostasis can be achieved for a liver defect, and the wound can be completely sealed, with an operation time of 15 s.

### Embodiment 19

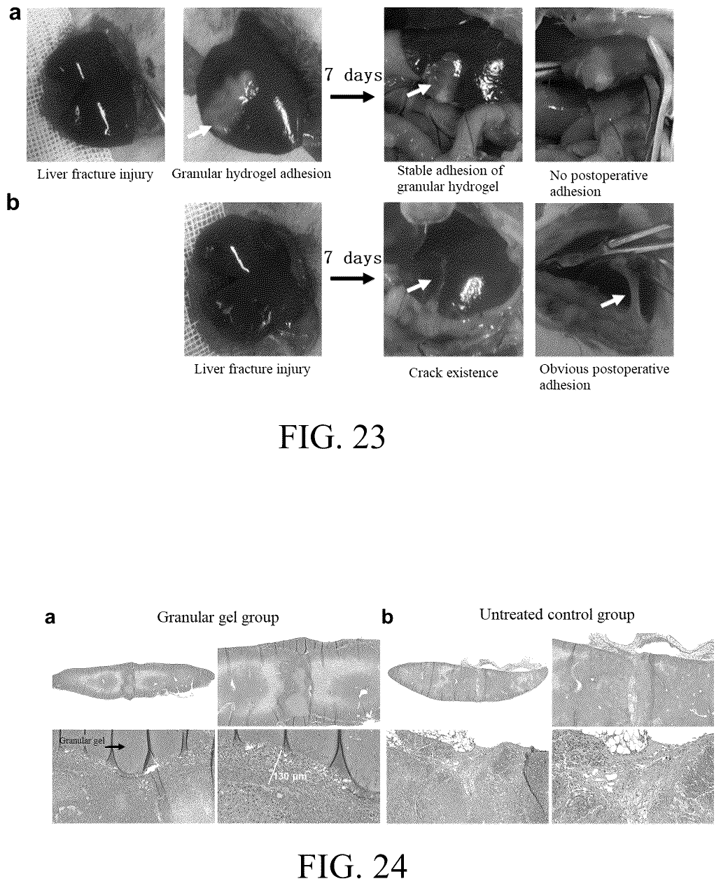

A linear wound with a length of 10 mm was made in a rat liver. The covalently crosslinkable gelatin granular gels prepared in Embodiments 1-4 were used for test. With the granular gel prepared in Embodiment 1 as an example, 1 mL of deionized water, 0.13 g of methacrylate gelatin colloidal granules and 0.005 g of Lap ultraviolet initiator were blended to obtain a gelatin granular gel. The gelatin granular gel was injected into the 10 mm linear wound of the mouse liver. After standing for 30 sec, the gelatin granular gel in the wound area was irradiated with an ultraviolet light source for 10 s to induce covalent crosslinking on surfaces between the granules and stably adhere with wound tissue to form a barrier. 7 days after the wound sealed, post-operative liver tissue was observed. As shown in FIG. 23 panel a, the gel can be stably bonded to the liver wound, and the liver wound does not form adhesion to the surrounding tissue. FIG. 24 panel a shows a hematoxylin-eosin (HE) staining of the linear injury of the liver tissue, it can be seen that the gelatin granular gel is stably bonded to the liver tissue, and the liver tissue at the linear defect is well healed without adhesion to the surrounding tissue.

### Comparative example 9

A linear wound with a length of 10 mm was made in a rat liver, and simple hemostasis was performed by using medical gauze. 7 days after the wound sealed, post-operative liver tissue was observed. As shown in FIG. 23 panel b, the liver tissue forms severe adhesion with the surrounding tissue. FIG. 24 panel b shows a histological staining, it can be seen that the liver tissue has a non-healing gap and has significant adhesion with the surrounding tissue.

1. A non-covalent bond and covalent bond two-field coupling crosslinked, injectable, moldable and printable granular hydrogel material, wherein a gelatin granule is used as a basic structural unit to form a continuous and porous granular network by means of reversible non-covalent bond crosslinking and covalent bond crosslinking between the gelatin granules, and the gelatin granules form the continuous and porous granular network by means of reversible self-assembly under the action of the non-covalent bonds so as to achieve injectable, printable, moldable and self-healing properties and the high-strength granular hydrogel material is further formed by initiating covalent bond crosslinking, wherein the gelatin granules have a size ranging from 20 nm to 50 μm, and a volume fraction of the gelatin granules in a total volume of the granular hydrogel material is 2 v/v % to 100 v/v %, a degree of substitution of covalent crosslinking groups on gelatin macromolecular chains in the gelatin granules is 5% to 80%, the continuous and porous granular network has a pore size of 0.1 μm to 100 μm and is formed by connecting granules through polymer chains, and the obtained granular hydrogel material has a compressive elastic modulus of 0.5 kPa-500 kPa.

2. A non-covalent bond and covalent bond two-field coupling crosslinked, injectable, moldable and printable granular hydrogel material of a core-shell structure, wherein a granule of a core-shell structure is used as a basic structural unit to form a continuous and porous granular network by means of reversible non-covalent bonds and covalent bonds between the granules, the gelatin granules of a core-shell structure form the continuous and porous granular network by means of reversible self-assembly under the action of the non-covalent bonds so as to achieve injectable, printable and moldable properties, and the high-strength granular hydrogel is further formed by initiating covalent bond crosslinking on surfaces of the granules to enhance curing, wherein shell layer granules of the granules of a core-shell structure have a size ranging from 50 nm to 50 μm and core layer granules of that have a size ranging from 10 nm to 1 μm, a volume fraction of the granules of a core-shell structure in a total volume of the granular hydrogel material is 2 v/v % to 100 v/v %, the obtained continuous and porous granular network has a pore size of 0.1 μm to 100 μm, and the obtained granular hydrogel material has an elastic modulus of 10 kPa to 1000 kPa.

3. A preparation method of the non-covalent bond and covalent bond two-field coupling crosslinked, injectable, moldable and printable granular hydrogel material according to claim 1, when covalent bond is crosslinked by free radical polymerization of granule surface groups, the preparation method comprises the following steps of:
(1) dissolving gelatin in an aqueous solution at 30° C. to 60° C. to obtain a gelatin aqueous solution with a concentration of 0.1 w/v % to 10 w/v %;
(2) adding a compound reacting with hydroxyl and amino groups on a gelatin polymer chain into the gelatin aqueous solution to obtain a modified gelatin polymer compound of formula III, wherein the compound reacting with the hydroxyl and amino groups on a gelatin polymer chain is a compound shown as formula I or formula II, preferably acrylic anhydride, acryloyl chloride, methacrylic anhydride, methacryloyl chloride, ethyl acrylic anhydride, ethyl acryloyl chloride, hydroxy acrylic anhydride, hydroxy acryloyl chloride, isobornyl acrylic anhydride, isobornyl acryloyl chloride, allyl isocyanate anhydride and allyl isocyanate chloride;
wherein
in formula I, formula II and formula III, R and R1 are selected from the group consisting of hydrogen, halogen atom, hydroxyl, sulfhydryl, amine group, nitro group, cyano group, aldehyde group, keto group, ester group, amide group, phosphonic acid group, phosphonate group, sulfonic acid group, sulfonate group, sulfone group, sulfoxide group, aryl group, and alkyl group;
(3) adding a polar organic solvent into the modified gelatin polymer compound solution until a modified gelatin polymer compound is precipitated out, followed by washing the modified gelatin polymer compound and re-dissolving with an aqueous solution at 30° C. to 60° C. to obtain a modified gelatin aqueous solution with a concentration of 0.1 w/v % to 20 w/v %;
(4) adjusting a pH value of the modified gelatin aqueous solution to 1-5 or 9-14, and dripping the polar organic solvent into the modified gelatin aqueous solution to obtain a covalently crosslinkable gelatin granular suspension, wherein a volume of the added polar organic solvent is 1 time to 10 times of a volume of the modified gelatin aqueous solution; carrying out crosslinking reaction for 1 hour to 12 hours at a normal temperature to obtain a covalently crosslinkable gelatin granular dispersion liquid after washing, and freeze-drying the granular dispersion liquid to obtain a modified gelatin granular powder; and
(5) blending the modified gelatin granular powder with the aqueous solution to obtain a colloidal gel, and adding a chemical initiator or a photo-crosslinking agent to initiate free radical polymerization, so that the modified gelatin granules are covalently crosslinked to further obtain mechanically-enhanced non-covalent bond and covalent bond composite crosslinked gelatin granules assembled to form the granular hydrogel material.

4. A preparation method of the non-covalent bond and covalent bond two-field coupling crosslinked, injectable, moldable and printable granular hydrogel material according to claim 1, when covalent bond is crosslinked by click chemistry of granule surface groups, the preparation method comprises the following steps of:
(1) dissolving gelatin in an aqueous solution at 30° C. to 60° C. to obtain a gelatin aqueous solution with a concentration of 0.1 w/v % to 10 w/v %;
(2) adding a compound capable of carrying out amidation reaction with carboxyl group or amino group on a surface of gelatin into the gelatin solution to respectively obtain a solution containing modified gelatin polymer compound A or a solution containing modified gelatin polymer compound B with a structural formula according with any one of formulas VI or VII, wherein the compound capable of carrying out amidation reaction with carboxyl or amino on the surface of gelatin is preferably a compound shown as chemical formulas IV or V, and is preferably azide succinimide/alkyne ethylamine, azide imine/propargylamine, mercaptoethylamine/ethyleneimine, and 2-amino ethanethiol/ethyleneimine; and in formula IV, formula V, formula VI and formula VII, R2 is a combination of azide/alkyne, sulfhydryl/double bond, thiol/alkene or diene/mono olefinic bond, and R3 is selected from the group consisting of hydrogen, halogen atom, hydroxyl, sulfhydryl, amine group, nitro group, cyano group, aldehyde group, keto group, ester group, amide group, phosphonic acid group, phosphonate group, sulfonic acid group, sulfonate group, sulfone group, sulfoxide group, aryl group and alkyl group;
(3) adding a polar organic solvent into each of the solution containing modified gelatin polymer compound A and the solution containing modified gelatin polymer compound B until a modified gelatin polymer compound is precipitated out, followed by washing the modified gelatin polymer compound and re-dissolving with an aqueous solution at 30° C. to 60° C. to obtain two modified gelatin aqueous solutions with a concentration of 0.1 w/v % to 20 w/v %, respectively;
(4) adjusting a pH value of each of the two modified gelatin aqueous solutions to 1-5 or 9-14, and dripping the polar organic solvent into each of the two modified gelatin aqueous solutions to obtain two covalently crosslinkable gelatin granular suspensions, wherein a volume of the added polar organic solvent is 1 time to 10 times of a volume of the modified gelatin aqueous solution; carrying out crosslinking reaction for 1 hour to 12 hours at a normal temperature to obtain two click chemical crosslinkable gelatin granular dispersion liquids after washing, and separately freeze-drying the two granular dispersion liquids to obtain two modified gelatin granular powders A and B; and
(5) uniformly blending the two gelatin granular powders A and B having click chemical group combinations at a ratio of 0.1-10, followed by rapidly blending with an aqueous solution uniformly to obtain a colloidal gel and stand for 2 min to 60 min to obtain the covalent crosslinked granular hydrogel material by means of covalent crosslinking between the gelatin granules in a click chemistry reaction.

5. A preparation method of the non-covalent bond and covalent bond two-field coupling crosslinked, injectable, moldable and printable granular hydrogel material of a core-shell structure according to claim 2, when covalent bond is crosslinked by free radical polymerization of granule surface groups, the preparation method comprises the following steps of:
(1) dissolving gelatin in an aqueous solution at 30° C. to 60° C. to obtain a gelatin aqueous solution with a concentration of 0.1 w/v % to 10 w/v %;
(2) adding a compound reacting with hydroxyl and amino groups on a gelatin polymer chain into the gelatin aqueous solution to obtain a modified gelatin polymer solution containing a modified gelatin polymer compound of formula III, wherein the compound reacting with the hydroxyl and amino groups on a gelatin polymer chain is preferably a compound shown as formula I or formula II;
wherein
in formula I, formula II and formula III, R and R1 are selected from the group consisting of hydrogen, halogen atom, hydroxyl, sulfhydryl, amine group, nitro group, cyano group, aldehyde group, keto group, ester group, amide group, phosphonic acid group, phosphonate group, sulfonic acid group, sulfonate group, sulfone group, sulfoxide group, aryl group and alkyl group;
(3) adding a polar organic solvent into the modified gelatin polymer compound solution until a modified gelatin polymer compound is precipitated out, followed by washing the modified gelatin polymer compound and re-dissolving with a suspension containing 0.1 w/v % to 50 w/v % of rigid nanoparticles at 30° C. to 60° C. to obtain a modified gelatin/rigid nanoparticle suspension with a concentration of 0.1 w/v % to 10 w/v % of gelatin capable of free radical polymerization crosslinking;
(4) adjusting a pH value of the modified gelatin/rigid nanoparticle suspension to 1-5 or 9-14, and dripping the polar organic solvent into the modified gelatin/rigid nanoparticle suspension to obtain a modified core-shell structural gelatin granular suspension, wherein a volume of the added polar organic solvent is 1 time to 10 times of a volume of the modified gelatin/rigid nanoparticle suspension, and carrying out crosslinking reaction for 1 hour to 12 hours at a normal temperature to obtain a modified gelatin core-shell granular dispersion liquid after washing, and freeze-drying the granular dispersion liquid to obtain a modified gelatin core-shell granular powder; and
(5) blending the modified gelatin core-shell granular powder with the aqueous solution, to obtain a colloidal gel, and adding a chemical initiator or a photo-crosslinking agent to initiate free radical polymerization, so that the modified gelatin granules are covalently crosslinked to further obtain mechanically-enhanced non-covalent bond and covalent bond composite crosslinked gelatin granules of a core-shell structure assembled to form the granular hydrogel material.

6. A preparation method of the non-covalent bond and covalent bond two-field coupling crosslinked, injectable, moldable and printable granular hydrogel material of a core-shell structure according to claim 2, when covalent bond is crosslinked by click chemistry of granule surface groups, the preparation method comprises the following steps of:
(1) dissolving gelatin in an aqueous solution at 30° C. to 60° C. to obtain a gelatin aqueous solution with a concentration of 0.1 w/v % to 10 w/v %;
(2) adding a compound capable of carrying out amidation reaction with carboxyl group or amino group on a surface of gelatin into the gelatin solution to respectively obtain a solution containing modified gelatin polymer compound C or a solution containing modified gelatin polymer compound D with a structural formula according with any one of formulas VI or VII, wherein the compound capable of carrying out amidation reaction with carboxyl or amino on the surface of the gelatin is preferably a compound shown as formula IV or V;
wherein
in formula IV, formula V, formula VI, and formula VII, R2 is a combination of azide/alkyne, sulfhydryl/double bond, thiol/alkene or diene/mono olefinic bond, and R3 is selected from the group consisting of hydrogen, halogen atom, hydroxyl, sulfhydryl, amine group, nitro group, cyano group, aldehyde group, keto group, ester group, amide group, phosphonic acid group, a phosphonate group, sulfonic acid group, sulfonate group, sulfone group, sulfoxide group, aryl group and alkyl group;
(3) adding a polar organic solvent into each of the solution containing modified gelatin polymer compound C and the solution containing modified gelatin polymer compound D until a modified gelatin polymer compound is precipitated out, followed by washing the modified gelatin polymer compound and re-dissolving with a suspension containing 0.1 w/v % to 50 w/v % of rigid nanoparticles at 30° C. to 60° C. to obtain two modified gelatin/rigid nanoparticle suspensions with a click chemical crosslinkable gelatin concentration of 0.1 w/v % to 10 w/v % respectively;
(4) adjusting a pH value of each of the two modified gelatin/rigid nanoparticle suspensions to 1-5 or 9-14, and dripping the polar organic solvent into each of the two modified gelatin/rigid nanoparticle suspensions to obtain two modified core-shell structural gelatin granular suspensions, wherein a volume of the added polar organic solvent is 1 time to 10 times of a volume of the modified gelatin/rigid nanoparticle suspension, and carrying out crosslinking reaction for 1 hour to 12 hours at a normal temperature to obtain two modified gelatin core-shell granular dispersion liquids after washing, and separately freeze-drying the granular dispersion liquids to obtain two modified gelatin core-shell granular powders C and D; and
(5) uniformly blending the two gelatin core-shell granular powders C and D having click chemical group combinations at a ratio of 0.1-10, followed by rapidly blending with an aqueous solution uniformly to obtain a colloidal gel and stand for 2 min to 60 min, and obtaining the covalent crosslinked granular hydrogel material by means of covalent crosslinking between the gelatin granules in a click chemistry reaction.

7. The preparation method according to claim 5, wherein the rigid nanoparticles are selected from at least one of silicon dioxide nanoparticles, lithium magnesium silicate nanoparticles, nano-clay particles, hydroxyapatite nanoparticles, iron oxide magnetic nanoparticles, barium titanate nanoparticles, graphene nanosheets, carbon nanotubes, bioglass nanoparticles, black phosphorus nanosheets, silk fibroin nanoparticles, polylactic acid nanoparticles, polyethylene nanoparticles, and polystyrene nanoparticles, wherein the rigid nanoparticles have a size ranging from 10 nm to 50 μm.

8. The preparation method according to claim 3, wherein
in step (3), the polar organic solvent is methanol, ethanol, isopropanol, butanol, acetone, acetonitrile or tetrahydrofuran; the aqueous solution is a solution containing a bioactive substance, and the bioactive substance is vitamins, amino acids, mineral elements, microecological regulators, growth factors or blood;
in step (5), the aqueous solution is directly blended with at least one of rigid granules of hydroxyapatite, silicon dioxide, bioglass, manganese dioxide, carbon quantum dots, graphene, montmorillonite, black phosphorus, silk fibroin and polylactic acid, wherein the rigid granules have a size ranging from 10 nm to 1 μm.

9. The granular hydrogel material according to claim 1 serving as a carrier or scaffold of a medicine component, which is used for repairing and filling wounds or defects of bone tissue, cartilage tissue, muscle and blood vessels, wherein the medicine component is at least one of vitamins, amino acids, mineral elements, microecological regulators, growth factors, protein macromolecular medicines, protein micromolecular medicines and living cells.

10. An application of the granular hydrogel material according to claim 1 as a bone repair filler material, wherein during application, covalently crosslinkable gelatin colloidal granules formed under the action of non-covalent bonds are blended with an aqueous solution to obtain a granular gel having a mass fraction of 5%-50% and a volume fraction of 10%-120%, the granular gel is directly injected into a bone defect area, and then a high-strength bone filling material is obtained by initiating covalent crosslinking between the gelatin colloidal granules.

11. An application of the granular hydrogel material according to claim 1 as a bioprinting ink for living cell-laden printing, wherein during application, covalently crosslinkable gelatin colloidal granules formed under the action of non-covalent bonds are blended with an aqueous solution to obtain a granular gel having a mass fraction of 5%-50% and a volume fraction of 10%-120%, then the granular gel is mixed with a cell suspension to obtain a cell-laden granular gel having a volume fraction of 10%-100% as a bioprinting ink, the bioprinting ink is extruded or subject to 3D ink-jet printing to obtain a scaffold of a 3D structure, then a high-strength cell-laden printed scaffold is obtained by initiating covalent crosslinking between the gelatin colloidal granules after printing.

12. An application of the granular hydrogel material according to claim 1 as a tissue adhesive gel material, wherein the gelatin granules have a size less than 10 μm, and an adhesive strength between the granular hydrogel and tissue is 5 kPa-100 kPa; covalently crosslinkable gelatin granules or granules of a core-shell structure are blended with a photo-crosslinking agent and then injected into a tissue injury site in vivo, and covalent crosslinking between the gelatin granules is achieved by light irradiating a gel surface to generate covalent bond polymerization; alternatively, a chemical initiator is blended with a gelatin composite gel and then injected into a tissue injury site in vivo, then covalent crosslinking is achieved after 1 min-30 min, and a stable adhesion is formed due to a mechanical interlocking effect between the gel material and tissue; and
alternatively, click chemical crosslinkable gelatin granules or granules of a core-shell structure are blended with an aqueous solution and then directly injected into a tissue injury site in vivo, then click chemical covalent crosslinking is achieved after 1 min-30 min, and a stable adhesion is formed due to a mechanical interlocking effect with tissues.

13. An application of the granular hydrogel material according to claim 1 as a post-operative anti-adhesion gel, wherein an injectable tissue adhesive gel is injected into a post-operative anti-adhesion site, and the injectable tissue adhesive gel stably covers the injury site after covalent crosslinking, wherein the granular hydrogel acts as a barrier to effectively prevent adhesion between tissue after surgery.

14. A rapid hemostatic sealant obtained by freeze-drying the gelatin granular suspension prepared according to claim 3, wherein the gelatin granular powder is uniformly blended with a chemical crosslinking agent powder or a photo-crosslinking agent powder, and then is directly sprayed on a bloody wound surface, covalent crosslinking between the gelatin granules is achieved by standing directly or photo-induced polymerization after the powder fully absorbs oozing blood; alternatively, the powder containing click chemical crosslinkable gelatin granules is blended and then directly sprayed on a bloody wound surface, and covalent crosslinking between the gelatin granules is achieved by standing directly after the powder fully absorbs oozing blood; and the granules form stable adhesion with tissue after crosslinking.

15. Applications of the granular hydrogel material according to claim 1 in preparation of a skin repairing material or medicine for post-operative wound surface sealing, in preparation of an oral ulcer material or medicine for post-operative wound surface sealing, in preparation of an intestinal leakage occlusion material or medicine for tissue fluid leakage occlusion, in preparation of a surgical suture material or medicine for tissue fluid leakage occlusion, in preparation of a liver hemostatic material or medicine, in preparation of a bone section hemostatic material or medicine, in preparation of an arterial hemostatic material or medicine, in preparation of a heart hemostatic material or medicine, in preparation of a cartilage repair material or medicine as tissue engineering scaffold material, in preparation of a bone repair material or medicine as tissue engineering scaffold material, and in preparation of a bone/cartilage composite defect repair material or medicine as tissue engineering scaffold material
